(19) United States
(12) Patent Application Publication
(10) Pub. No.: US 2018/0140550 A1
(43) Pub. Date: May 24, 2018

(54) CLEAVABLE POLYMERIC MICELLES

(71) Applicant: The Regents of the University of Michigan, Ann Arbor, MI (US)

(72) Inventors: Kanokwan Sansanaphongpricha, Ann Arbor, MI (US); Hongwei Chen, Ann Arbor, MI (US); Duxin Sun, Ann Arbor, MI (US)

(21) Appl. No.: 15/873,400

(22) Filed: Jan. 17, 2018

Related U.S. Application Data
(63) Continuation of application No. 14/838,618, filed on Aug. 28, 2015.
(60) Provisional application No. 62/043,648, filed on Aug. 29, 2014.

(51) Int. Cl. A61K 9/107; A61K 47/34; A61K 31/00; A61K 31/704

(52) U.S. Cl. CPC A61K 9/107; A61K 31/704; A61K 31/00; A61K 47/34

(57) ABSTRACT

The present invention provides compositions, systems, and methods employing cleavable polymeric micelles. For example, provided herein are compositions comprising micelles that contain a hydrophobic agent (e.g., metal nanoparticles and/or therapeutic agent), where the micelles are formed from a plurality of amphiphilic polymer molecules that comprise a hydrophilic polymer and a hydrophobic polymer, where the hydrophobic polymer comprises a cleavable Furan-Maleimide adduct. Also provided herein are methods of administering such compositions to a subject and treating a localized area of the subject with a device that emits heat, NIR light, and/or alternating magnetic current such that at least some of the micelles inside the subject near the localized area are disrupted (e.g., releasing a therapeutic agent).

The present application is a continuation of U.S. patent application Ser. No. 14/838,618, filed Aug. 28, 2015, which claims priority to U.S. provisional application Ser. No. 62/043,648 filed Aug. 29, 2014, both of which are herein incorporated by reference in their entireties.

### FIELD OF THE INVENTION

The present invention provides compositions, systems, and methods employing cleavable polymeric micelles. For example, provided herein are compositions comprising micelles that contain a hydrophobic agent (e.g., metal nanoparticles and/or therapeutic agent), where the micelles are formed from a plurality of amphiphilic polymer molecules that comprise a hydrophilic polymer and a hydrophobic polymer, where the hydrophobic polymer comprises a cleavable Furan-Maleimide adduct. Also provided herein are methods of administering such compositions to a subject and treating a localized area of the subject with a device that emits heat, NIR light, and/or alternating magnetic current such that at least some of the micelles inside the subject near the localized area are disrupted (e.g., releasing a therapeutic agent).

### BACKGROUND

Block copolymer micelles have long been studied yet still gain a lot of interest for drug delivery systems for a number of reasons. Polymeric micelles not only improve physicochemical properties of the loaded-drug but also control drug release over a period of time at a particular area (1). Several kinds of polymer have been investigated such as PLGA and PCL because of their biodegradability. However, the ability to control the drug release triggered by external stimuli needs to be improved. Triggered-responsive materials have recently attracted a great deal of attention from researchers (2, 3). These materials better control drug release at specific targets to maximize therapeutic outcomes and minimize adverse drug reactions from non-specific release. One of the most common methods to trigger drug release is to use temperature (4). Most traditional thermal sensitive polymers can undergo the structural change between hydrophilic and hydrophobic parts of their polymer. Some polymers have a lower critical solution temperature (LCST) such as Poly (N-isopropylacrylamide, PNIPAAM). They can undergo phase changes when heated above LCST leading to structural shrinkage and squeezing out of a small molecule drug. Whereas polymers that possess an upper critical solution temperature (UCST) can swell and become more hydrophilic when the temperature is above their UCST (2, 5, 6, 7, 8). However, the polymer backbone of these traditional thermal sensitive polymers fails to cleave resulting in the inability of releasing nanoparticles loaded inside the micelles. Those nanoparticles remain in big clusters, ≥100 nm in size and may obstruct deep tumor penetration (9, 10, 11). To overcome high interstitial pressure and dense collagen matrix in tumor, nanoparticles with the size smaller than 50 nm are necessary (12).

One of the most well-known reversible chemical reactions is Diels-Alder reaction. Diels-Alder (DA) and retro Diels-Alder (rDA) reactions were discovered in year 1928 in Germany by Professor Otto Diels and his student, Kurt Alder. This spectacular discovery resulted in the receipt of the Nobel Prize in Chemistry in 1950 (13). In 1994, Kuramoto et al made a hydrophobic polymer by using this reaction. They used difurfuryl adipate (DFA) for the furan source and used bismaleimido-diphenyl-methane (BMD) for the maleimide source (14; herein incorporated by reference). After that, the DA reaction has been intensive studied by using different structures of furan and maleimide molecules (15). However, the Diels-Alder reaction has not been widely used and has limited application because it requires a relatively high temperature to induce the reversible reaction. McElhanon et al, 2004 synthesized an easily removed surfactant by using 2-N-dodecyl hydrophobic furan and N-(4-hydroxyphynyl) hydrophilic maleimide (16). This surfactant was proved useful as removable templates for the construction of microporous materials. Yamashita, 2011 made use of the maleimide-modified polyethylene glycol (Mw 20,000) to conjugate with furfuryl disulfide-gold nanorods. The high temperature induces rDA leading to the release of polyethylene glycol from the gold nanorod surface (17).

### SUMMARY OF THE INVENTION

The present invention provides compositions, systems, and methods employing cleavable polymeric micelles. For example, provided herein are compositions comprising micelles that contain a hydrophobic agent (e.g., metal nanoparticles and/or therapeutic agent), where the micelles are formed from a plurality of amphiphilic polymer molecules that comprise a hydrophilic polymer and a hydrophobic polymer, where the hydrophobic polymer comprises a cleavable Furan-Maleimide adduct. Also provided herein are methods of administering such compositions to a subject and treating a localized area of the subject with a device that emits heat, NIR light, and/or alternating magnetic current such that at least some of the micelles inside the subject near the localized area are disrupted (e.g., releasing a therapeutic agent).

In some embodiments, provided herein are compositions comprising: a) an aqueous solution; b) at least one micelle, in the aqueous solution, which is formed from a plurality of amphiphilic polymer molecules, wherein each of the amphiphilic polymer molecules comprises a hydrophilic polymer and a hydrophobic polymer, and wherein the hydrophobic polymer comprises a first region, a second region, and a cleavable Furan-Maleimide adduct which separates the first from the second regions; and c) at least one hydrophobic agent which is located inside the at least one micelle.

In other embodiments, provided herein are systems and kits comprising: a) a plurality of amphiphilic polymer molecules, wherein each of the amphiphilic polymer molecules comprises a hydrophilic polymer and a hydrophobic polymer, wherein the hydrophobic polymer comprises a first region, a second region, and a cleavable Furan-Maleimide adduct which separates the first and second regions; and b) at least one hydrophobic agent.

In certain embodiments, provided herein are methods of making hydrophobic agent containing micelles comprising: a) mixing a plurality of hydrophobic agents with a plurality of amphiphilic polymer molecules to generate an initial solution, wherein each of the amphiphilic polymer molecules comprises a hydrophilic polymer and a hydrophobic polymer, wherein the hydrophobic polymer comprises a first region, a second region, and a cleavable Furan-Maleimide adduct which separates the first and second regions; and b) transferring the initial solution (e.g., drop-wise or other suitable method) into an aqueous solution such that a plurality of micelles are formed, wherein at least a portion of the plurality of micelles contain at least one of the hydrophobic agents.

In additional embodiments, provided herein are methods of treating a subject comprising: a) administering a composition to a subject, wherein the composition comprises a plurality of micelles that are each formed from a plurality of amphiphilic polymer molecules and which contain at least one hydrophobic metal nanoparticle, wherein each of the amphiphilic polymer molecules comprises a hydrophilic polymer and a hydrophobic polymer, and wherein the hydrophobic polymer comprises a first region, a second region, and a cleavable Furan-Maleimide adduct which separates the first and second regions; and b) contacting a localized area (or non-localized area) of the subject with a device that can emit electromagnetic radiation, wherein the contacting with the device cleaves at least some of the cleavable Furan-Maleimide adducts thereby disrupting at least some of the micelles inside the subject that are near the localized area of the subject.

In further embodiments, provided herein are methods of generating single-dispersed single metal nanoparticle containing micelles comprising: subjecting a metal nanoparticle containing micelle (MNM) to electromagnetic radiation such that a plurality of single-dispersed single metal nanoparticle containing micelles (SDSMNs) are generated, wherein the MMN comprises a plurality of amphiphilic polymer molecules and contains a plurality of hydrophobic metal nanoparticles, wherein each of the amphiphilic polymer molecules comprises a hydrophilic polymer and a hydrophobic polymer, and wherein the hydrophobic polymer comprises a first region, a second region, and a cleavable Furan-Maleimide adduct which separates the first and second regions, and wherein each of the SDSMNs comprises: i) a cleaved portion of the amphiphilic polymer, wherein the cleaved portion comprises the hydrophilic polymer and the second region of the hydrophobic polymer (e.g., containing a Maleimide compound), but does not contain the first region of the hydrophobic polymer; and ii) a single hydrophobic metal nanoparticle.

In additional embodiments, provided herein are methods of generating Janus nanoparticles comprising: a) subjecting a composition to electromagnetic radiation, wherein the composition comprises a plurality of first nanoparticle containing micelles (FNMs) and a plurality of second nanoparticle containing micelles (SNMs), wherein each of the FNMs comprises a plurality of first amphiphilic polymer molecules and a plurality of first hydrophobic nanoparticles, wherein each of the SNMs comprises a plurality of second amphiphilic polymer molecules and a plurality of second hydrophobic nanoparticles that: i) are composed of a different material than the first hydrophobic nanoparticles, and/or ii) have an average size that is smaller than the average size of the first hydrophobic nanoparticles, wherein each of the first and second amphiphilic polymer molecules comprise a hydrophilic polymer and a hydrophobic polymer, wherein the hydrophobic polymer comprises a first region, a second region, and a cleavable Furan-Maleimide adduct which separates the first and second regions, wherein the subjecting the composition to the electromagnetic radiation causes the Furan-Maleimide adducts to be cleaved thereby generating cleaved portions of the amphiphilic polymer molecules, and wherein each of the cleaved portions comprises the hydrophilic polymer and the second region of the hydrophobic polymer (e.g., containing a Maleimide compound), but does not contain the first region of the hydrophobic polymer; and b) incubating the composition such that a plurality of Janus nanoparticles form, wherein the Janus nanoparticles comprise a plurality of the first hydrophobic nanoparticles, a plurality of the second hydrophobic nanoparticles, and a plurality of the cleaved portions.

In certain embodiments, provided herein are methods of generating Janus nanoparticles comprising: a) subjecting a composition to electromagnetic radiation (e.g., heat), wherein the composition comprises: i) a plurality of seed micelles, ii) a plurality of first nanoparticle containing micelles (FNMs), and iii) a plurality of second nanoparticle containing micelles (SNMs), wherein each of the seed micelles comprises a plurality of first amphiphilic polymer molecules, wherein each of the FNMs comprises a plurality of second amphiphilic polymer molecules and a plurality of first hydrophobic nanoparticles, wherein each of the SNMs comprises a plurality of third amphiphilic polymer molecules and a plurality of second hydrophobic nanoparticles that: i) are composed of a different material than the first hydrophobic nanoparticles, and/or ii) have an average size that is smaller than the average size of the first hydrophobic nanoparticles, wherein each of the first, second, and third amphiphilic polymer molecules comprise a hydrophilic polymer and a hydrophobic polymer, wherein the hydrophobic polymer comprises a first region, a second region, and a cleavable Furan-Maleimide adduct which separates the first and second regions, wherein the subjecting the composition to the electromagnetic radiation causes the Furan-Maleimide adducts to be cleaved in some of the first, second, and third amphiphilic polymer molecules, thereby generating a plurality of cleaved portions of the amphiphilic polymer molecules, and wherein each of the cleaved portions comprise the first region of the hydrophobic polymers, but does not contain the hydrophilic polymer or the second region of the hydrophobic polymer; and b) incubating the composition such that a plurality of Janus nanoparticles form, wherein the Janus nanoparticles comprise: i) a plurality of the first hydrophobic nanoparticles, ii) a plurality of the second hydrophobic nanoparticles, iii) a plurality of the cleaved portions, and iv) a plurality of the first, second, and/or third amphiphilic polymer molecules (e.g., as shown in FIG. 16). In certain embodiments, provided herein are compositions comprising the Janus nanoparticles generated by this method. In other embodiments, such Janus nanoparticles further comprise a therapeutic agent.

In certain embodiments, provided herein are methods of generating ball-like micelles comprising: a) subjecting a composition to electromagnetic radiation, wherein the composition comprises: i) a plurality of seed micelles, and ii) a plurality of nanoparticle containing micelles (NMs), wherein each of the seed micelles comprises a plurality of first amphiphilic polymer molecules, wherein each of the NMs comprises a plurality of second amphiphilic polymer molecules and a plurality of hydrophobic nanoparticles, wherein each of the first and second amphiphilic polymer molecules comprise a hydrophilic polymer and a hydrophobic polymer, wherein the hydrophobic polymer comprises a first region, a second region, and a cleavable Furan-Maleimide adduct which separates the first and second regions, wherein the subjecting the composition to the electromagnetic radiation causes the Furan-Maleimide adducts to be cleaved in some of the first and second amphiphilic polymer molecules, thereby generating a plurality of cleaved portions of the amphiphilic polymer molecules, and

wherein each of the cleaved portions comprise the first region of the hydrophobic polymer, but does not contain the hydrophilic polymer or the second region of the hydrophobic polymer; and b) incubating the composition such that a plurality of ball-like micelles form, wherein the ball-like micelles comprise: i) a plurality of the first hydrophobic nanoparticles, ii) a plurality of the cleaved portions, and iii) a plurality of the first and/or second amphiphilic polymer molecules.

In certain embodiments, provided herein are compositions comprising the ball-like micelles generated by this method. In other embodiments, such ball-like micelles further comprise a therapeutic agent.

In certain embodiments, the first and second (and/or third) amphiphilic polymers are different. In other embodiments, the electromagnetic radiation is selected from the group consisting of: thermal radiation, infrared radiation, visible light, X-rays, radio waves, microwaves, ultraviolet radiation, and gamma rays. In further embodiments, the electromagnetic radiation comprises heat. In certain embodiments, the plurality of second hydrophobic metal nanoparticles have an average size (e.g., diameter) of about 1 nm, 2 nm, 3 nm, 4 nm, 5 nm . . . 10 nm . . . 15 nm . . . 25 nm . . . 50 nm . . . or 100 nm). In other embodiments, the plurality of first hydrophobic metal nanoparticles have an average size (e.g., diameter) of about 1 nm, 2 nm, 3 nm, 4 nm, 5 nm . . . 10 nm . . . 15 nm . . . 25 nm . . . 50 nm . . . or 100 nm). In other embodiments, the first and second hydrophobic nanoparticles comprise different metals or different materials.

In additional embodiments, the different metals are selected from gold and iron. In additional embodiments, the different materials are selected from iron oxide, gold, quantum dots, or polymeric materials.

In some embodiments, provided herein are methods of treating or detecting disease comprising: administering the Janus nanoparticles described herein to a patient such that a disease is at least partially treated and/or detected.

In other embodiments, provided herein are systems comprising: a) the Janus nanoparticles described herein, and b) a device configured to generate the electromagnetic radiation.

In further embodiments, provided herein are methods of generating ball-like micelles comprising: a) subjecting a composition to electromagnetic radiation, wherein the composition comprises a plurality of metal nanoparticle containing micelles (MNMs) and a plurality of hydrophobic agent containing micelles (HAMs), wherein each of the MNMs comprises a plurality of first amphiphilic polymer molecules and a plurality of metal hydrophobic nanoparticles, wherein each of the HAMs comprises a plurality of second amphiphilic polymer molecules and a plurality of hydrophobic agents, wherein each of the first and second amphiphilic polymer molecules comprise a hydrophilic polymer and a hydrophobic polymer, wherein the hydrophobic polymer comprises a first region, a second region, and a cleavable Furan-Maleimide adduct which separates the first and second regions, wherein the subjecting the composition to the electromagnetic radiation causes the Furan-Maleimide adducts to be cleaved thereby generating cleaved portions of the amphiphilic polymer molecules, and wherein each of the cleaved portions comprises the hydrophilic polymer and the second region of the hydrophobic polymer, but does not contain the first region of the hydrophobic polymer; and b) incubating the composition such that a plurality of ball-like micelles form, wherein the ball-like micelles comprise a plurality of the metal nanoparticles arranged in a hollow ball-like structure, a plurality of hydrophobic agents located inside the hollow ball-like structure, and a plurality of the cleaved portions.

In further embodiments, the first and second amphiphilic polymers are different. In additional embodiments, the electromagnetic radiation is selected from the group consisting of: thermal radiation, infrared radiation, visible light, X-rays, radio waves, microwaves, ultraviolet radiation, and gamma rays. In additional embodiments, the electromagnetic radiation comprises heat. In further embodiments, the hydrophobic agent comprises an MRI dye or a therapeutic agent. In additional embodiments, the metal nanoparticles comprise iron or gold. In some embodiments, the hydrophobic agent comprises IR820, IR780, or a hydrophobic drug.

In particular embodiments, provided herein are methods of treating or detecting disease comprising: administering the ball-like Micelles described herein to a patient such that a disease is at least partially treated and/or detected.

In other embodiments, provided herein are systems comprising: a) the ball-like Micelles described herein, and b) a device configured to generate the electromagnetic radiation.

In certain embodiments, the electromagnetic radiation is selected from the group consisting of: thermal radiation, infrared radiation, visible light, X-rays, radio waves, microwaves, ultraviolet radiation, and gamma rays. In further embodiments, the electromagnetic radiation is provided by a device selected from the group consisting of: a near-infrared light generating device, a heat source device, and a device that generates an alternating magnetic current. In additional embodiments, the MNN micelle contains ≥2 metal nanoparticles (e.g., 2 . . . 5 . . . 10 . . . 15 . . . 20, or more nanoparticles).

In particular embodiments, each of the plurality of micelles further contains at least one therapeutic agent. In additional embodiments, the contacting releases the therapeutic agent from the micelles that are disrupted. In other embodiments, the localized area of the subject comprises a tumor or other disease site. In certain embodiments, the subject is a human or animal (e.g., dog, cat, horse, cow, pig, etc.). In further embodiments, the device comprises a NIR laser or NIR LED source. In certain embodiments, the near-infrared light has a wavelength in the range from 700 nm to 2500 nm (e.g., about 700 nm . . . 800 nm . . . 900 nm . . . 1000 nm . . . 1500 nm . . . 1750 nm . . . 2000 nm . . . and 2500 nm). In particular embodiments, the heat provided by the device is about 50-110 degrees Celsius (e.g., 50 . . . 65 . . . 80 . . . 90 . . . and 110 degrees Celsius).

In some embodiments, the at least one hydrophobic agent comprises one or more metal nanoparticles (e.g., iron oxide, gold, copper, silver, titanium (e.g., titanium oxide), zinc, cobalt, cerium oxide, aluminum, magnesium, etc.). In further embodiments, the metal nanoparticles comprise an organic hydrophobic coating. In further embodiments, the at least one hydrophobic agent comprises one or more therapeutic agents or diagnostic agents. In other embodiments, the at least one hydrophobic agent comprises at least one therapeutic agent (and/or at least one diagnostic agent) and at least one metal nanoparticle. In further embodiments, the one or more therapeutic agents are anti-cancer agents, or one or more diagnostic agents are near-infrared dyes (e.g., for NIR imaging such as IR 820, indocyanine green, etc.; see Luo et al., Biomaterials. 2011 October; 32(29):7127-38 herein incorporated by reference for such dyes) for cancer diagnosis. In particular embodiments, the aqueous solution comprises a physiologically tolerable buffer.

In certain embodiments, the at least one micelle comprises a plurality of micelles, and wherein the plurality of micelles are single-dispersed in the aqueous solution. In other embodiments, the at least one micelle comprises a plurality of micelles, and wherein the plurality of micelles are ball-like micelles characterized by a hollow core (see FIG. 9).

In other embodiments, the hydrophilic polymer comprises Thiol methoxy polyethylene oxide. In particular embodiments, the hydrophilic polymer may comprise molecules selected from polyalkylene oxides, polyols, poly(oxyalkylene)-substituted diols and polyols, polyoxyethylated sorbitol, polyoxyethylated glucose, poly(acrylic acids) and analogs and copolymers thereof, polymaleic acids, polyacrylamides, poly(olefinic alcohols), polyethylene oxides, poly(N-vinyl lactams), polyoxazolines, polyvinylamines, and copolymers thereof, polyethylene glycol, poly(ethylene oxide)-poly(propylene oxide) copolymers, glycerol, polyglycerol, propylene glycol, mono-, di- and tri-polyoxyethylated glycerol, mono- and di-polyoxyethylated propylene glycol, mono- and di-polyoxyethylated trimethylene glycol, poly(acrylic acid), poly(methacrylic acid), poly(hydroxyethylmethacrylate), poly(hydroxyethylacrylate), poly(methylalkylsulfoxide acrylates), poly(methylalkylsulfoxide methacrylates), polyacrylamide, poly(methacrylamide), poly(dimethylacrylamide), poly(N-isopropylacrylamide), and copolymers thereof, poly (vinyl alcohols) and copolymers thereof, poly(vinyl pyrrolidones), poly(vinyl caprolactams), and copolymers thereof, poly(methyloxazoline) and poly(ethyloxazoline).

In some embodiments, the hydrophobic polymer comprises the DA-b-PEO polymer shown in FIG. 1. In certain embodiments, the hydrophobic polymer comprises molecules selected from the group consisting of: polystyrenes, styrene-butadiene copolymers, polystyrene-based elastomers, polyethylenes, polypropylenes, polytetrafluoroethylenes, extended polytetrafluoroethylenes, polymethylmetacrylates, ethylene-co-vinyl acetates, polymethylsiloxane, polyphenylmethylsiloxanes, modified polysiloxanes, polyethers, polyurethanes, polyether-urethanes, polyethylene terephthalates, polysulphones, polyglycolide, poly dl-polylactide, poly d-lactide, poly l-lactide, polydioxanone, polytrimethylenecarbonate, polyorthocarbonates, polyanhydride, proteins, carboxylated polysaccharides, aminated polysaccharides, aliphatic polyesters, polyhydroxyalkanoates, polyothroesters, polyurethanes, polyanhydrides, ellulosic ethers, cellulosic esters, zein, shellac, gluten, polylactide, hydrophobic starch derivatives, polyvinyl acetate polymers, polymers or copolymers derived from an acrylic acid ester and/or a methacrylic acid ester and combinations thereof, polyurethanes, acrylic polymers, epoxies, silicones and fluorosilicones.

In additional embodiments, the therapeutic agent is a drug, a vitamin, a nutritional supplement, a cosmeceutical, or a mixture thereof. In other embodiments, the therapeutic agent is a polyfunctional hydrophobic drug, a lipophilic drug, a pharmaceutically acceptable salt, isomer or derivative thereof, or a mixture thereof. In particular embodiments, the therapeutic agent is selected from the group consisting of analgesics, anti-inflammatory agents, anthelmintics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-Blockers, cardiac inotropic agents, corticosteroids, diuretics, anti-parkinsonian agents, gastrointestinal agents, histamine H, receptor antagonists, keratolytics, lipid regulating agents, anti-anginal agents, nutritional agents, opioid analgesics, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof.

In certain embodiments, the therapeutic agent is tramadol, celecoxib, etodolac, refocoxib, oxaprozin, leflunomide, diclofenac, nabumetone, ibuprofen, flurbiprofen, tetrahydrocannabinol, capsaicin, ketorolac, albendazole, ivermectin, amiodarone, zileuton, zafirlukast, albuterol, montelukast, azithromycin, ciprofloxacin, clarithromycin, dirithromycin, rifabutine, rifapentine, trovafloxacin, baclofen, ritanovir, saquinavir, nelfinavir, efavirenz, dicoumarol, tirofibran, cilostazol, ticlidopine, clopidrogel, oprevelkin, paroxetine, sertraline, venlafaxine, bupropion, clomipramine, miglitol, repaglinide, glymepride, pioglitazone, rosigiltazone, troglitazone, glyburide, glipizide, glibenclamide, carbamezepine, fosphenytion, tiagabine, topiramate, lamotrigine, vigabatrin, amphotericin B, butenafine, terbinafine, itraconazole, flucanazole, miconazole, ketoconazole, metronidazole, griseofulvin, nitrofurantoin, spironolactone, lisinopril, benezepril, nifedipine, nilsolidipine, telmisartan, irbesartan, eposartan, valsartan, candesartan, minoxidil, terzosin, halofantrine, mefloquine, dihydroergotamine, ergotamine, frovatriptan, pizofetin, sumatriptan, zolmitriptan, naratiptan, rizatriptan, aminogluthemide, busulphan, cyclosporine, mitoxantrone, irinotecan, etoposide, teniposide, paclitaxel, tacrolimus, sirolimus, tamoxifen, camptothecan, topotecan, nilutanide, bicalutanide, pseudo-ephedrine, toremifene, atovaquone, metronidazole, furazolidone, paricalcitol, benzonatate, midazolam, zolpidem, gabapentin, zopiclone, digoxin, beclomethsone, budesonide, betamethasone, prednisolone, cisapride, cimetidine, loperamide, famotidine, lanosprazole, rabeprazole, nizatidine, omeprazole, citrizine, cinnarizine, dexchlopheniramine, loratadine, clemastine, fexofenadine, chlorpheniramine, acutretin, tazarotene, calciprotiene, calcitriol, targretin, ergocalciferol, cholecalciferol, isotreinoin, tretinoin, calcifediol, fenofibrate, probucol, gemfibrozil, cerivistatin, pravastatin, simvastatin, fluvastatin, atorvastatin, tizanidine, dantrolene, isosorbide dinatrate, a carotene, dihydrotachysterol, vitamin A, vitamin D, vitamin E, vitamin K, an essential fatty acid source, codeine, fentanyl, methadone, nalbuphine, pentazocine, clomiphene, danazol, dihydro epiandrosterone, medroxyprogesterone, progesterone, rimexolone, megesterol acetate, osteradiol, finasteride, mefepristone, amphetamine, L-thryroxine, tamsulosin, methoxsalen, tacrine, donepezil, raloxifene, vertoporfin, sibutramine, pyridostigmine, a pharmaceutically acceptable salt, isomer, or derivative thereof, or a mixture thereof.

### DESCRIPTION OF THE FIGURES

FIGS. 1A-B show the synthesis scheme for the thermo-cleavable polymer, DA-b-PEO.

FIG. 1C shows an NMR spectrum of the DA-b-PEO polymer.

FIG. 2A shows various NMR spectrum that show that the hydrophobic polymer backbone (DA) cleavage. FIG. 2B shows that after the hydrophobic part (DA) of thermo-cleavable polymer are exposed to 100° C. for an hour, the percent of the cycloadduct reduces from 68.08% to 11.11%, which is relatively close to the percent of the cycloadduct of the freshly prepared hydrophobic polymer.

FIG. 3 shows a schematic that demonstrates the use of DA-b-PEO as a coating material for IONPs or micelle formation.

FIG. 4 shows a chart that demonstrates no significant difference of temperature generation from 15 nm IONP-Dox loaded thermo-cleavable micelles (TCM) and 15 nm-IONP-Dox loaded non-thermo cleavable micelles (non-TCM), or control micelles, at the same iron oxide concentration, 0.2 mg/ml. FIG. 4 shows that IONPs act as a photothermal mediator converting NIR light to heat.

FIGS. 5A-B show that both Dox-IONP TCM and non-TCM are stable at 37° C. FIG. 5A shows that there is no aggregate formed after 2 hours of 37° C. exposure (A left). In contrast, as shown in FIG. 5A right, after 2 hours of 80° C. treatment, Dox-IONP TCM are ruptured and release the payload as the big aggregates are obviously formed. FIG. 5B shows that, after NIR laser trigger, Dox-IONP loaded TCM form big aggregates similar to the heat treatment at 80° C., while there is no significant change in Dox-IONP loaded non-TCM.

FIG. 6A shows the percent Dox released at 80 degrees Celsius, which shows that TCM can release Dox 3 times higher than non-TCM after 80° C. treatment for 60 minutes.

FIG. 6B shows the percent Dox released after NIR laser irradiation for 24 minutes, showing that, with NIR laser treatment, Dox can release from TCM 4 times higher than non-NIR treatment, and 2 times higher than the control micelles with NIR laser treatment.

FIG. 7 shows TEM image of the Dox-IONPs loaded thermo-cleavable micelles before (A), after temperature trigger at 80° C. (B), and NIR laser irradiation (C). FIG. 7A shows that IONPs form micelle-like clusters. In contrast, after 80° C. or NIR laser exposure, Dox-IONPs loaded thermo-cleavable micelles loss the micelle-like structure and become single-dispersed IONPs as shown in FIGS. 7B and 7C. FIG. 7D shows Dox-IONPs loaded non-thermocleavable micelles, control micelles. However, non-TCM remain their micelle-like structure after 80° C. (7E) or NIR laser exposure (7F).

FIG. 8 shows a schematic picture depicting tumor penetration of thermocleavable Dox-IONPs micelles. Dox-IONPs TCM remain stable in the blood stream and reach the tumor sites by enhanced permeability and retention effect (EPR). NIR laser then triggers the dissociation of the micelles by inducing reverse Diels-Alder (rDA) reaction and cleaving the polymer backbone resulting in the release of both Dox and 15 nm IONPs as single IONPs. These smaller diameter IONPs have a better tumor penetration because they are able to pass through dense extracellular matrix. The deeper tumor penetration brings about more effective cancer therapy.

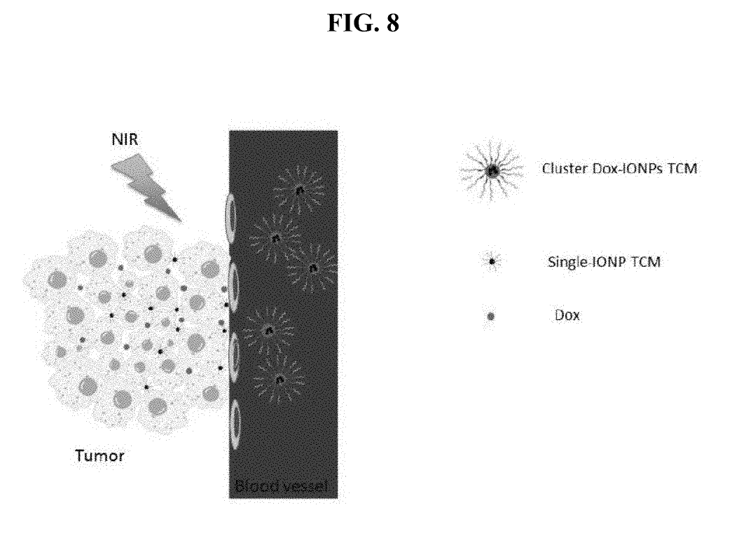

FIG. 9 shows a schematic picture representing the transformation process from the cluster IONP-loaded micelles into single-dispersion and ball-liked structure micelles.

FIGS. 10A-C show three different structures of IONPs micelles. FIG. 10A shows the cluster IONPs micelles (i) before heat exposure, single-dispersed IONPs (ii) after heat exposure without additional DA-b-PEO polymer, and ball-like structure with the hollow core (iii) after heat exposure with additional excessive DA-b-PEO polymer. The top row are images from a conventional TEM and the bottom row are images from STEM respectively. FIG. 10B shows STEM image of the ball-like micelles that have IONPs align as a ring and have a hollow core. FIG. 10C show the density profile of ball-like micelles is higher at the edge of the micelles but low at the center of the micelles.

FIG. 11A shows that DiI-IONP ball-like micelles are formed from the combination of two different encapsulated particles in TCM micelles. IONPs-loaded TCM micelles are mixed with DiI-loaded TCM micelles (DiI is a dye with CAS number 41085-99-8) in an aqueous media and subsequently exposed to heat treatment. IONPs form a ball-like structure with encapsulated DiI dye inside the core of the ball-like micelles.

FIG. 11B shows as chart that shows the percent of DiI dye in supernatant measured by UV absorbance from the solution shown in the lower panel. The lower left picture demonstrates that DiI dye molecules are encapsulated within the ball-like structure as the DiI dye precipitate down together with IONPs after centrifugation at high speed. In contrast, the mixture of DiI-loaded non-TCM and IONPs-loaded non-TCM cannot form the DiI-IONPs loaded ball structure even after heat treatment. Each kind of micelle is still in water separately because the IONPs-loaded non TCM precipitate down as can be seen by the black pallets at the bottom of the tube, while the DiI-loaded non TCM are still suspended in water as can be seen in the pink solution. The lower right picture shows that without heat exposure, DiI-IONPs ball-like structure cannot be effectively formed from TCM and cannot be formed at all from non-TCM.

FIG. 12A shows a schematic picture describing an exemplary process for making Janus nanoparticles using mixtures of cleavable micelles. 15 nm IONPs TCM are mixed with 5 nm IONPs TCM and heated up to 94° C. for 2 hours. After the hydrophobic polymer backbone is cleaved by the heat, the two kinds of TCM combine together and generate Janus nanoparticles that generally have 15 nm IONPs on one side/part and 5 nm IONPs on the other side/part.

FIG. 12B shows TEM images that demonstrate 15 nm and 5 nm IONPs both in TCM and non-TCM original cluster before heat treatment. However, after being exposed to high temperature, TCM create a new type of micelles, which have both 15 nm and 5 nm IONPs in the same micelles. In contrast, after heat treatment, 15 nm and 5 nm IONP non-TCM are still in separate micelles as the original micelle solution. This confirms that Janus nanoparticles are formed due to the use of the cleavable backbone.

FIG. 12C show an image of the Janus nanoparticles at high magnification. The 15 nm IONPs are deposited on the left side of the ball and 5 nm IONPs are deposited at the other.

FIGS. 13A-C show (A) A synthesis scheme of DA-b-PEO amphiphilic diblock thermo-cleavable copolymer. An equimolar of DFA and BMD was mixed in tetrachloro ethane and the reaction was carried out at 70° C. for 7 days. The molecular weight of the polymer was 5,090 Da. Then SH-mPEG was conjugated with the maleimide terminus of the hydrophobic backbone via Michael addition and yielded the final product with the molecular weight of 9,800 Da. (B) a cartoon picture represent the thermo-cleavable polymer and the hydrophobic backbone cleavage after high temperature exposure. (C)¹H NMR of the hydrophobic backbone at different time points and temperatures: freshly prepared (top), 48 hours after 70° C. heat treatment (middle), and 1 hour after 100° C. heat treatment. It clearly shows that the cycloadducts peaks at 3.09 and 5.32 ppm increase after polymerization via Diels-Alder reaction at 70° C. for 48 hours. However, these peaks disappear after the temperature increases to 100° C. for an hour. This indicates the cycloadduct disruption via retro Diels-Alder.

FIG. 14. TEM images of the original FeTCM (a) and AuTCM (b) before heat treatment. (c) A TEM image of multi-building block Au/IONP JNS after self-assembly process. (d) A high magnification TEM image and a cartoon picture show an asymmetrical structure of JNS. (e) A STEM-HADDF image of JNS and (f) XEDS element maps of JNS confirm an asymmetrical pattern of JNS. (g) TEM, (h) STEM, and (i) XEDS images of scramble dodecenethiol-coated AuNPs and oleic-coated IONPs loaded in TCM show a random pattern of Au/IONP mixture in micelles.

FIGS. 15 (a) and (e) represent TEM images of FeBNS and AuBNS after self-assembly of FeTCM and AuTCM respectively. FIGS. 15 (b) and (f) demonstrate STEM-HADDF images of FeBNS and AuBNS at low magnifications. FIGS. 15 (c) and (g) are STEM-HADDF images of FeBNS and AuBNS at high magnifications with a color heat map. FIGS. 15 (d) and (h) show density profiles of FeBNS and AuBNS.

FIG. 16 shows a schematic diagram that demonstrates a proposed transformation mechanism from cluster micelles to multi-building block Janus or ball-like nanostructures. First, a FeTCM collides with free TCM seed. Simultaneously, another AuTCM can also collide with the same seed from the opposite direction and subsequently fuse together resulting in self-reorganization to form JNS. If only a kind of NP-TCMs is used, BNS will be formed instead of JNS.

### DEFINITIONS

As used herein, “single-dispersed,” in reference to metal nanoparticles, means that the metal nanoparticles are separately suspended in aqueous media and neither clump together, nor physically form aggregates.

As used herein, a “Furan-Maleimide adduct” is shown in the schematic below and can be formed in a Diels-Alder reaction between a compound with a furan end group and a compound with a maleimide end group.

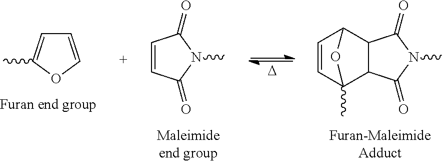

### DETAILED DESCRIPTION

The present invention provides compositions, systems, and methods employing cleavable polymeric micelles. For example, provided herein are compositions comprising micelles that contain a hydrophobic agent (e.g., metal nanoparticles and/or therapeutic agent), where the micelles are formed from a plurality of amphiphilic polymer molecules that comprise a hydrophilic polymer and a hydrophobic polymer, where the hydrophobic polymer comprises a cleavable Furan-Maleimide adduct. Also provided herein are methods of administering such compositions to a subject and treating a localized area of the subject with a device that emits heat, NIR light, and/or alternating magnetic current such that at least some of the micelles inside the subject near the localized area are disrupted (e.g., releasing a therapeutic agent).

In certain embodiments, provided herein methods for Diels-Alder amphiphilic block copolymer synthesis and its applications for (1) coating nanoparticles and micelle formation, (2) controlled drug and nanoparticle release, and (3) controlled transformation process—single and ball-like structure of iron oxide micelles. With the use of cleavable amphiphilic block copolymer, one is able to transform the cluster nanoparticles encapsulated in the micelles to smaller size of single-dispersed nanoparticles and control drug release simultaneously. The single-dispersed nanoparticles, for example, allow for deep tumor or other tissue penetration. In certain embodiments, the micelles with the ball-like structure provides the benefit of, for example, a high drug loading because they have more void space inside the micelles compared to other kind of micelles.

As described in Example 1 below, iron oxide nanoparticles (IONPs) are used as a photothermal mediator to convert near-infrared light (NIR) to heat (see, e.g., 21, 19). The heat subsequently breaks apart the polymer backbone via retro Diels-Alder reaction (rDA) (see, e.g., 22, 23) resulting in the release of both the nanoparticles and a small molecule drug. Doxorubicin (Dox) is also encapsulated into the thermo-cleavable micelles together with IONPs. Dox was chosen as a model drug because it has been used in clinic for cancer treatment. During the process of transformation, Dox can also be released out of the micelles. This demonstrates that the thermo-cleavable polymeric micelles can generate both single-dispersed nanoparticles and control drug release at the same time leading to deeper tumor penetration and better therapeutic outcomes. Moreover, general production of individual-dispersed IONPs is difficult to control, and requires multi-steps (24, 25). Nonetheless, with the cleavable amphiphilic block copolymers described herein, the transformation of the cluster IONPs can be simply used to make single-dispersed IONPs micelles stable in aqueous solution. This can be applied, for example, to the process of single-dispersed IONPs production in aqueous at industrial level because of the ease of scale up, and reproducibility.

The present disclosure is not limited by the type of metal nanoparticles that are employed. In certain embodiments, iron oxide nanoparticles are employed. Iron oxide nanoparticles (IONPs) have long been used for magnetic resonance imaging (MRI), hyperthermia, and photothermal therapy (PTT) (18, 19) because of their unique properties and safety. IONPs have capability of reducing T2 relaxation providing contrast images for the tumor areas and they also generate high temperature under alternating magnetic field or NIR light treatment (20). With these properties, IONPs could be used as diagnostic and PTT agents for cancer treatment.

In certain embodiments, the thermo-cleavable micelles described herein can be used to control both small molecule drug and nanoparticle release by using the external triggers such as high temperature and NIR laser light. This controlled drug release can mitigate premature release and enhance drug accumulation at the tumor site. Moreover, the unique property of the thermo-cleavable polymers (e.g., DA-b-PEO copolymer) allows one to control the morphology of the nanocomposites. In particular embodiments, the single-dispersed IONPs can be easily produced by treating the original cluster IONP micelles with high temperature. This method provides a benefit over the traditional method as the reaction happens in aqueous solution. The single-dispersed IONPs also have the advantage for PTT in terms of deep tumor penetration due to a smaller diameter. Furthermore, the excessive addition of the thermo-cleavable polymer in the system allows one to make the ball-like IONP micelles. This structure has a bigger void volume, so it can entrap a higher amount of drug and nanoparticles, which benefits cancer therapy and yield a better therapeutic outcome.

### EXAMPLES

### Example 1

### Cleavable Amphiphilic Block Copolymer Synthesis and Characterization

This Example describes the synthesis and characterization of a cleavable amphiphilic block copolymer, and its use to form micelles containing metal nanoparticles and therapeutic agents which can be disrupted with NIR (near infra-read) light treatment.

### Materials and Methods

### Materials:

furfuryl alcohol (98%), triethanolamine (TEA, 99%), dioxane (99.5%, extra dry), and 1,1,2,2 tetrachloro ethane (TCE, 98.5%) were purchased from Acros Organics. Petroleum ether (certified ACS grade), and dichloromehane (certified ACS grade) were purchased from Fisher Scientific. Ethyl acetate (anhydrous, 99.8%), tetrahydrofuran (THF, anhydrous 99.8%), adipoyl chloride, bismaleimido diphynyl methane (BMD), and dimethyl sulfoxide (DMSO, 99.5%) were purchased from Sigma-Aldrich. Thiol methoxy polyethylene oxide 5 KDa was purchased from NanoCS. Doxorubicin HCl (99.5%) was purchased from Polymed therapeutics. Polystyrene-b-polyethylene oxide (Ps-b-PEO), Mw 10,300 Da used for the control micelles was purchased from Polymer Source.

### Synthesis of IONPs:

15 nm IONPs were synthesized by using previously reported in the literature (19). Briefly, a mixture of 0.890 g FeO(OH), 19.8 g oleic acid and 25.0 g 1-octadecene in a three-neck flask was heated under stirring to 200° C. under N2, 30 minutes later the temperature was set at 220° C. for 1 h, then the temperature was increased gradually to 310° C. (20° C./5 minutes) and kept at this temperature for 1 hour. The solution became black when the temperature was increased to 320° C. and kept at this temperature for 1 hour. After the reaction was completed, the reaction mixture was cooled and the nanocrystals were precipitated by adding chloroform and acetone.

### Synthesis of Difurfuryl Adipate (DFA):

difurfuryl adipate was synthesized by using the previously published method by Kuramoto, 1994 (14, herein incorporated by reference). Briefly, adipoyl chloride was added dropwise to furfuryl alcohol in cold dioxane. The reaction continues at 0° C. for 3 hours. The product was purified by column chromatography using petroleum ether and ethyl acetate (2:1) as a mobile phase. The final product was viscous brown liquid and the structure was confirmed by using H¹NMR spectroscopy.

### Synthesis of Cleavable Hydrophobic Backbone Polymer, Diels-Alder Polymer (DA):

the DA polymer was synthesized from difufuryl adipate (DFA) and bismaleimido diphenyl methane (BMD) monomer as reported by Gandini, 2009 (28, herein incorporated by reference). An equimolar of DFA and BMD was mixed in TCE and the reaction continued at 70° C. for 9 days. The final product was precipitated in petroleum ether and characterized by using H¹NMR spectroscopy.

### Synthesis Thermo-Cleavable Polymer (DA-b-PEO) Via Michael Addition:

the excess molar concentration of thiol-methoxy polyethylene oxide, molecular weight 5,000 Da (SH-mPEO), was added into the solution of DA polymer in DCM with a few drops of TEA. The reaction continued overnight and the product was precipitated in petroleum ether. The polymer structure was confirmed by using H¹NMR spectroscopy and molecular weight was determined by using GPC.

### Cycloadduct Conversion:

in order to determine the percent of cycloadduct conversion, DFA and BMD were reacted at 70° C. for 48 hours to induce Diels-Alder reaction. While retro Diels-Alder happened at 100° C. leads to the cycloadduct cleavage. The percent of cycloadduct conversion was calculated from the area under the peaks appeared in H¹ NMR at 7.5 ppm and 5.3 ppm, which indicates furan ring in the starting material and cycloadduct in the product respectively. % Conversion (29)={AUC at 5.3 ppm/[AUC at 5.3 ppm+AUC at 7.5 ppm]}×100

### IONPs-Loaded and Dox-IONPs Loaded Micelles Formation:

for IONPs-loaded micelles, 4 mg of 15 nm IONPs were mixed with 40 mg of DA-b-PEO in 4 ml THF. Then the solution was transferred dropwise into 40 ml water under vigorous agitation. The solution was open to the air overnight to evaporate THF. IONP-loaded micelles were purified by centrifugation twice to get rid of free micelles. For Dox-IONPs loaded micelles, Dox.HCl was deprotonated overnight with TEA (1:2 molar ratio) in DMSO to get the hydrophobic Dox (30). Then 4 mg of hydrophobic Dox was mixed with IONPs and DA-b-PEO respectively in THF. A similar method with making IONP-loaded micelles and purification were used for formulating Dox-IONP loaded micelles. Polystyrene-b-polyethylene oxide (PS-b-PEO) was used for making non-cleavable control micelles. Encapsulation and loading efficiency of IONPs and Dox were determined by UV spectrophotometry.

### Photothermal Effect Determination:

0.2 mg/ml 15 nm IONPs were used for generation of the photothermal effect from both thermo-cleavable micelles and the control micelles. 200 ul of each sample were put on 96-well plate and were exposed to the NIR laser 885 nm, 2.5 W/cm² with 5×8 mm spot size. Phosphate buffer was used as a control. The temperature was measured by thermal camera.

### Dox Release Determination:

After the samples were either heated at 80° C. or exposed to NIR light, the released Dox was extracted by using 200 ul of chloroform. Subsequently, the chloroform layer were taken and evaporated overnight. Dox powder was reconstituted in DMSO and the amount of released Dox was measured by UV spectroscopy.

### Results

### Synthesis of the Thermo-Cleavable Polymer, DA-b-PEO.

DA-b-PEO polymer has molecular weight (Mw) 8,850 Da, Polydispersity index (PDI) 1.458. The molecular weight of the hydrophobic part (DA) of the polymer is 5,090 Da and the molecular weight of the hydrophilic part of the polymer is 5,000 Da. Mw and PDI were measured by gel permeation chromatography (GPC) as shown in table 1.

TABLE 1

| Polymer | Mw (Da) | Mn (Da) | PDI |
|---|---|---|---|
| DA | 5090 | 2418 | 2.100 |
| DA-b-PEO | 8850 | 6088 | 1.458 |

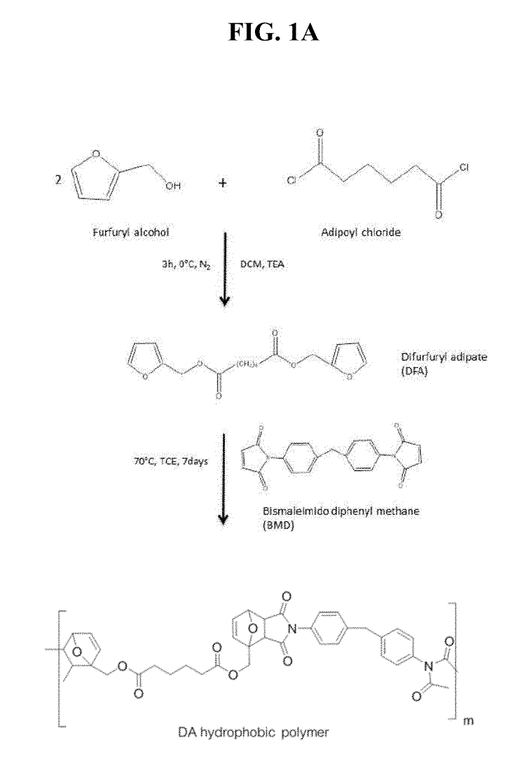

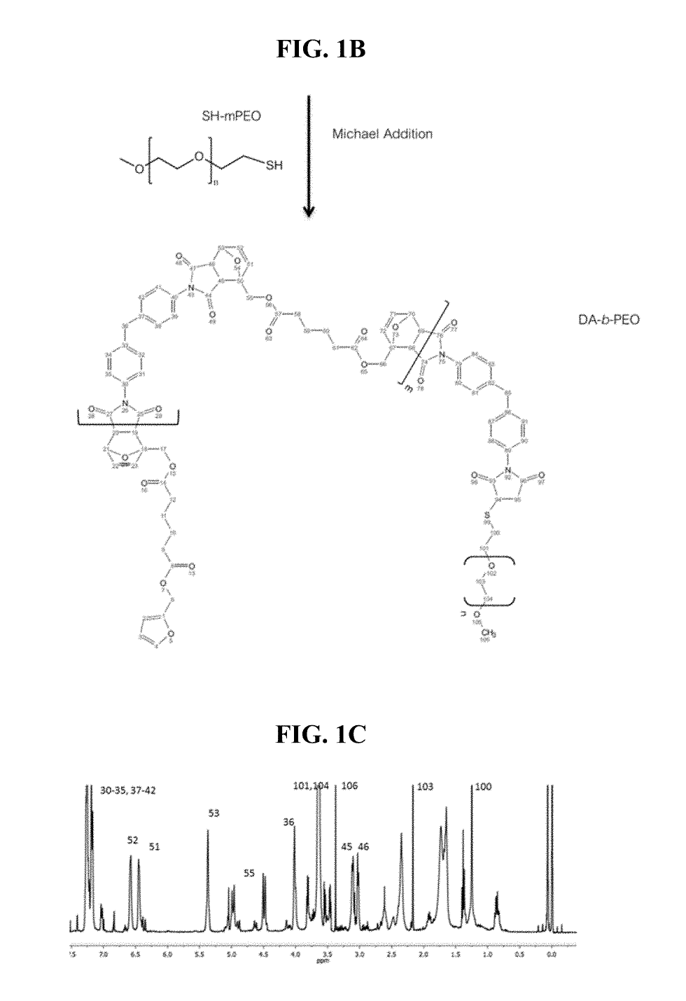

Diels-Alder hydrophobic polymer (DA) was synthesized as previous reported by Kuramoto, 1994 (14) with a modification. Then the hydrophilic polymer, Thiolated polyethylene oxide (SH-mPEO) was added to the DA polymer. The synthesis scheme is shown in FIG. 1A. Thiol functional group of SH-mPEO can react with the maleimide terminal end of the hydrophobic polymer via Michael addition (26, 27) and subsequently obtain a novel amphiphilic di block co-polymer called DA-b-PEO. The new peak (see NMR spectrum in FIG. 1B) emerged at 3.75 ppm indicates the successful synthesis of DA-b-PEO polymer as the hydrophobic backbone (DA) does not have this peak. These data also agree with the data from gel permeation chromatography (GPC), which shows that the Mw of the polymer increases from 5,090 Da to 8,850 Da.

### Evidence of Cleavable Backbone

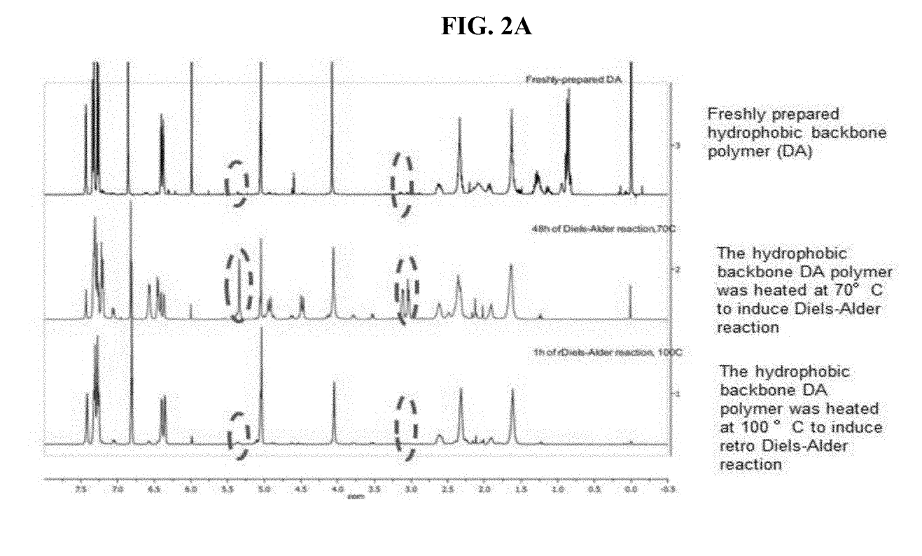

FIG. 2A shows various NMR spectrum that show that the hydrophobic polymer backbone (DA) cleavage. At 70° C., difurfuryl adipate (DFA) can react with bismaleimido diphenyl methane (BMD) via Diels-Alder reaction and form the cycloadduct structure as the peak shown in NMR spectrum at 3.09 and 5.31 ppm. Once the temperature increases to 100° C., retro Diels-Alder becomes predominant leading to the dissociation of the covalent bond in cycloadduct resulting in the reduction of the peak at 3.09 and 5.31 ppm. FIG. 2B shows that after the hydrophobic part (DA) of thermo-cleavable polymer are exposed to 100° C. for an hour, the percent of the cycloadduct reduces from 68.08% to 11.11%, which is relatively close to the percent of the cycloadduct of the freshly prepared hydrophobic polymer. It is concluded that the hydrophobic backbone of the thermo-cleavable polymer can be cleaved after being treated at 100° C. for an hour.

### Coating Material for IONPs and Micelle Formation

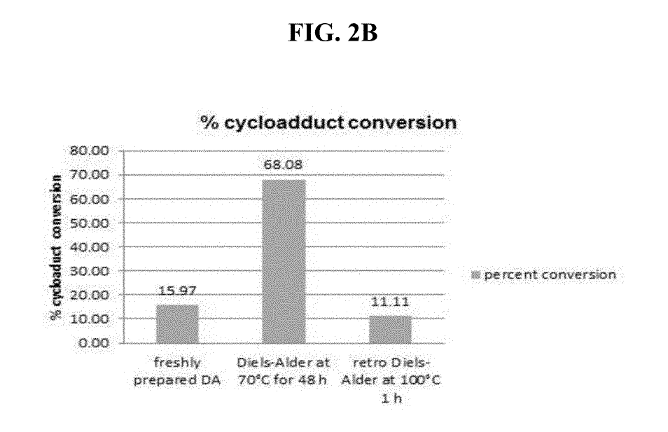

FIG. 3 shows a schematic that demonstrates the use of DA-b-PEO as a coating material for IONPs or micelle formation. To form the micelles, both hydrophobic (DA polymer) and hydrophilic (PEO) parts are used. The hydrophobic part forms the core, which can entrap hydrophobic molecules such as Dox and IONPs, and the hydrophilic part assemble as the shell, which helps increase solubility and prolong blood circulation time in the body. In this case, Doxorubicin and IONPs were incorporated into the thermo-cleavable micelles (Dox-IONP TCM) as a model drug and photothermal mediator for biomedical application respectively. Both 15 nm IONPs and Dox are spontaneously encapsulated into the hydrophobic core of the micelles. Dox-IONP loaded non-thermo-cleavable micelles (Dox-IONP non TCM) were produced with the similar method to Dox-IONP loaded TCM; however, PS-b-PEO was used instead of DA-b-PEO. Table 2 presents the size and polydispersity index (PDI) of three types of micelles.

TABLE 2

| | IONP | Dox-IONP loaded | Dox-IONP loaded |
|---|---|---|---|
| | loaded TCM | TCM | non TCM |
| Diameter (nm) | 58.77 | 37.84 | 68.06 |
| PDI | 0.222 | 0.518 | 0.042 |

Photothermal Effect from IONPs TCM

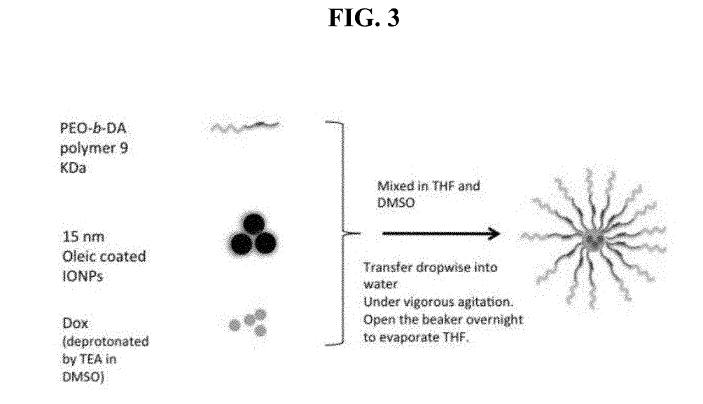

Data from the chart in FIG. 4 demonstrate no significant difference of temperature generation from 15 nm IONP-Dox loaded thermo-cleavable micelles (TCM) and 15 nm-IONP-Dox loaded non-thermo cleavable micelles (non-TCM), or control micelles, at the same iron oxide concentration, 0.2 mg/ml. In this case, IONPs act as a photothermal mediator converting NIR light to heat. TCM and non-TCM can reach 82.3° C. and 87.4° C. respectively, after 10 minutes of 885 nm NIR laser treatment with 2.5 W/cm2 of power. However, both kinds of micelles produce significantly higher temperature than phosphate buffer solution (PBS). This indicates that the thermo-cleavable polymer and the control polymer do not interfere the heat production from IONPs encapsulated in the micelles. Therefore, these micelles can be used as photothermal mediators for treatments, such as cancer hyperthermia treatment.

### Doxorubicin Release Triggered by Temperature and Near Infrared Laser Irradiation

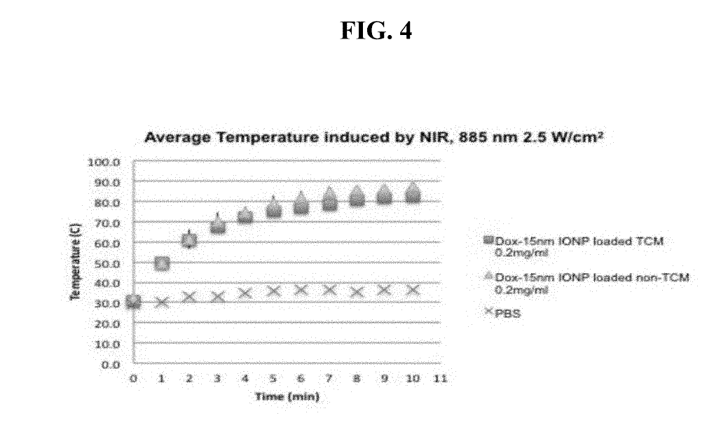

External triggers induce micelles dissociation for controlled drug release application. Both Dox-IONP TCM and non-TCM are stable at 37° C. This is shown in FIG. 5A, as there is no aggregate formed after 2 hours of 37° C. exposure (A left). In contrast, as shown in FIG. 5A right, after 2 hours of 80° C. treatment, Dox-IONP TCM are ruptured and release the payload as the big aggregates are obviously formed. The aggregates are the hydrophobic residues of the thermo-cleavable polymer, Dox, and unencapsulated IONPs. There are no aggregates formed from non-TCM even though they are exposed to the same temperature with the TCM (A right). Both Dox-IONP loaded TCM and non-TCM are also exposed to NIR laser for 24 minutes to examine the NIR-induced drug release. After NIR laser trigger, as shown in FIG. 5B, Dox-IONP loaded TCM form big aggregates similar to the heat treatment at 80° C., while there is no significant change in Dox-IONP loaded non-TCM. This indicates the non-TCM are neither sensitive to the high temperature nor NIR laser triggers as well as incapability of releasing the payload. It is explained that the TCM release Dox and IONPs by temperature-induced Diels-Alder reaction resulting in the cleavage of the cycloadduct in the hydrophobic backbone of the polymer. Moreover, this also indicates that both high temperature and NIR light can be used as external stimuli for controlled drug release from the TCM.

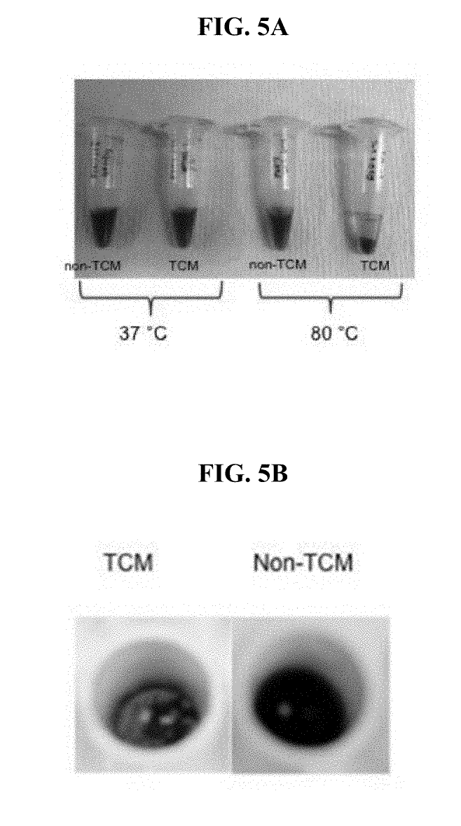

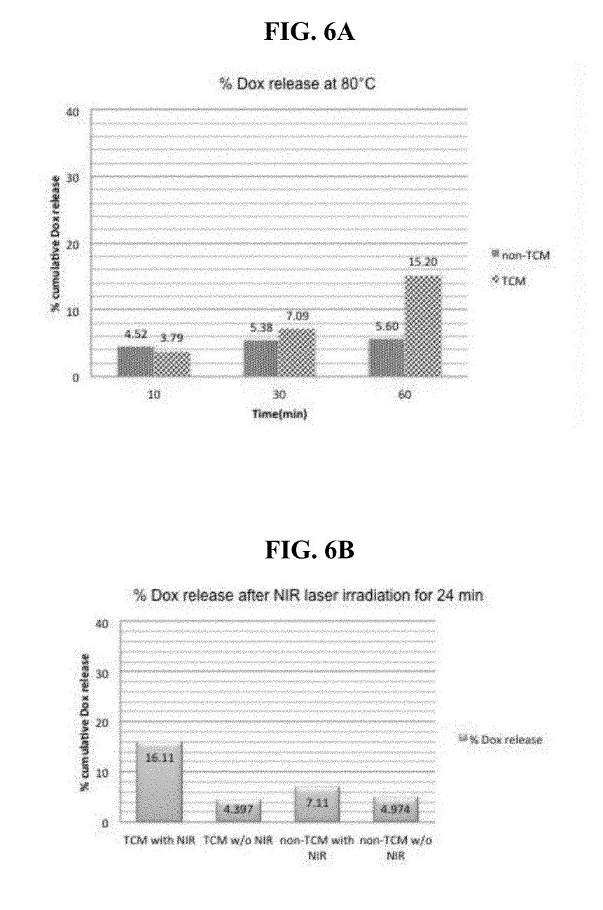

FIG. 6A shows that TCM can release Dox 3 times higher than non-TCM after 80° C. treatment for 60 minutes. This result agrees with the Dox release induced by NIR laser irradiation for 24 minutes as shown in FIG. 6B. It also demonstrates that with NIR laser treatment, Dox can release from TCM 4 times higher than non-NIR treatment, and 2 times higher than the control micelles with NIR laser treatment. This indicates that both high temperature, 80° C. and NIR laser irradiation can trigger Dox release from the thermo-cleavable micelles, while the non-cleavable micelles do not have significant difference in Dox release at 80° C. and NIR laser irradiation.

### IONPs Release Triggered by Temperature and Near Infrared Laser Irradiation

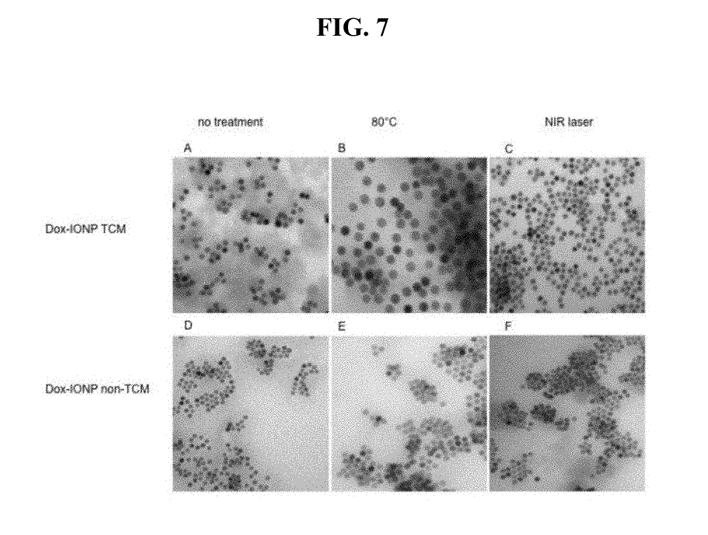

FIG. 7 shows TEM image of the Dox-IONPs loaded thermo-cleavable micelles before (A), after temperature trigger at 80° C. (B), and NIR laser irradiation (C). FIG. 7A shows that IONPs form micelle-like clusters. In contrast, after 80° C. or NIR laser exposure, Dox-IONPs loaded thermo-cleavable micelles loss the micelle-like structure and become single-dispersed IONPs as shown in FIGS. 7B and 7C. FIG. 7D shows Dox-IONPs loaded non-cleavable micelles, control micelles. However, non-TCM remain their micelle-like structure after 80° C. (7E) or NIR laser exposure (7F). This confirms that the TCM can be cleaved and reattach back to make the single-dispersed IONP micelles. This process facilitates the large scale production of the single-dispersed IONPs in aqueous solution.

### Morphology Transformation

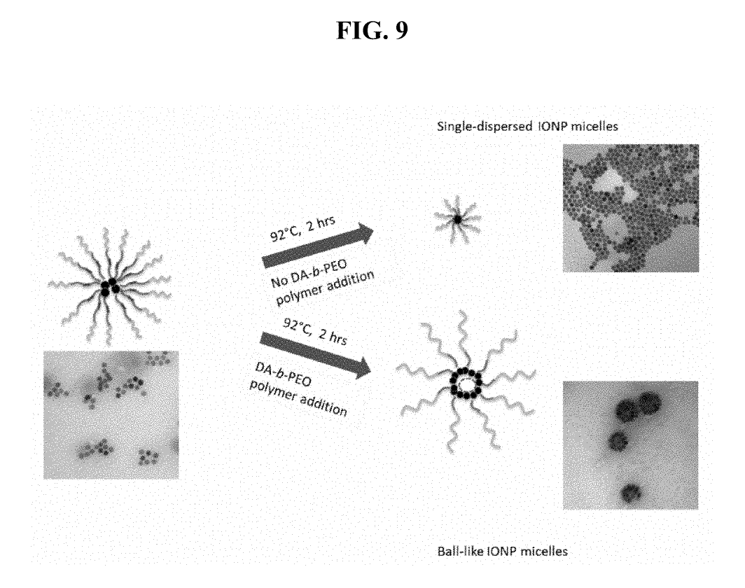

FIG. 9 shows a schematic picture representing the transformation process from the cluster IONP-loaded micelles into single-dispersion and ball-liked structure micelles. After cluster IONP-loaded micelles are exposed to high temperature, 92° C. for 2 hours, the polymer backbone is cleaved via retro Diels-Alder reaction resulting in release of IONPs. The residues of the polymer subsequently reattach and suspend the single IONPs in the solution. However, with the excessive additional DA-b-PEO polymer and heat treatment, the ball-like structure IONP micelles can be created. This structure has IONPs align at the periphery of the polymer, so the core of the micelles becomes hollow. The TEM pictures demonstrate that without the addition of the thermo-cleavable polymer (DA-b-PEO) to IONPs, single-dispersed ION micelles are generated. In contrast, the excessive amount of the polymer added to the system is sufficient to generate the ball-like structure IONP micelles. This indicates that the DA-b-PEO polymer possesses a unique property to regulate the process of morphology transformation. Moreover, different micelle morphologies have different biomedical applications. Whereas the single-dispersed IONP micelles have a smaller size resulting in better tumor penetration, the ball-liked IONP micelles can load the higher amount of hydrophobic drug inside the ball.

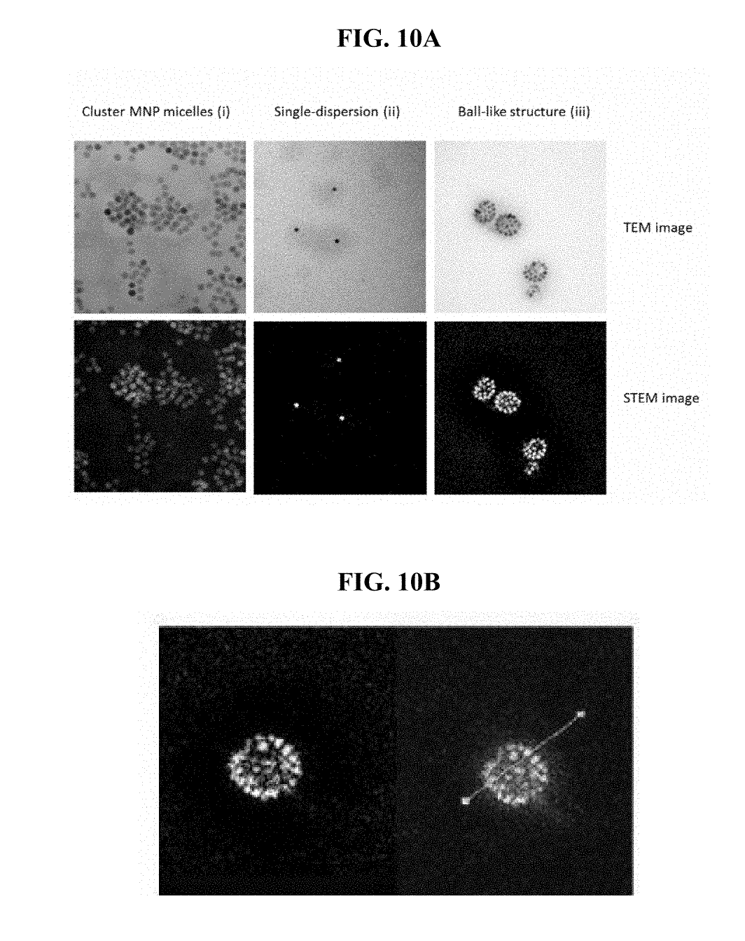

FIG. 10 shows three different structures of IONPs micelles. FIG. 10A demonstrates the cluster IONPs micelles (i) before heat exposure, single-dispersed IONPs (ii) after heat exposure without additional DA-b-PEO polymer, and ball-like structure with the hollow core (iii) after heat exposure with additional excessive DA-b-PEO polymer. The top row is the images from a conventional TEM and the bottom row is images from STEM respectively. The morphology of these three micelles is obviously different. FIG. 10B shows STEM image of the ball-like micelles that have IONPs align as a ring and have the hollow core. FIG. 10C show the density profile of ball-like micelles is higher at the edge of the micelles but low at the center of the micelles. These data agree with the image from STEM and TEM.

### Example 2

### Generating Ball-Like Structure and Janus Nanoparticles

This Example describes methods of generating ball-like structure by mixing two types of cleavable micelles, and methods of generating Janus nanoparticles.

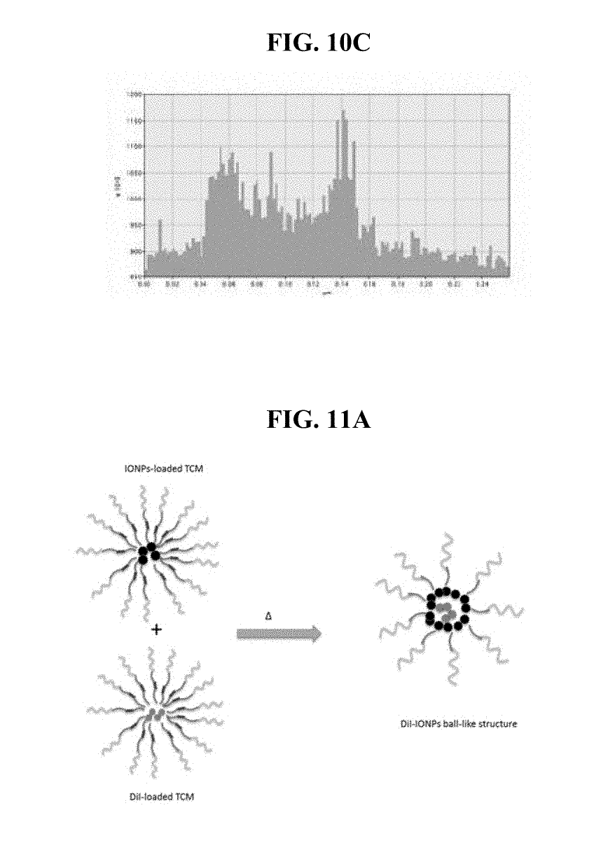

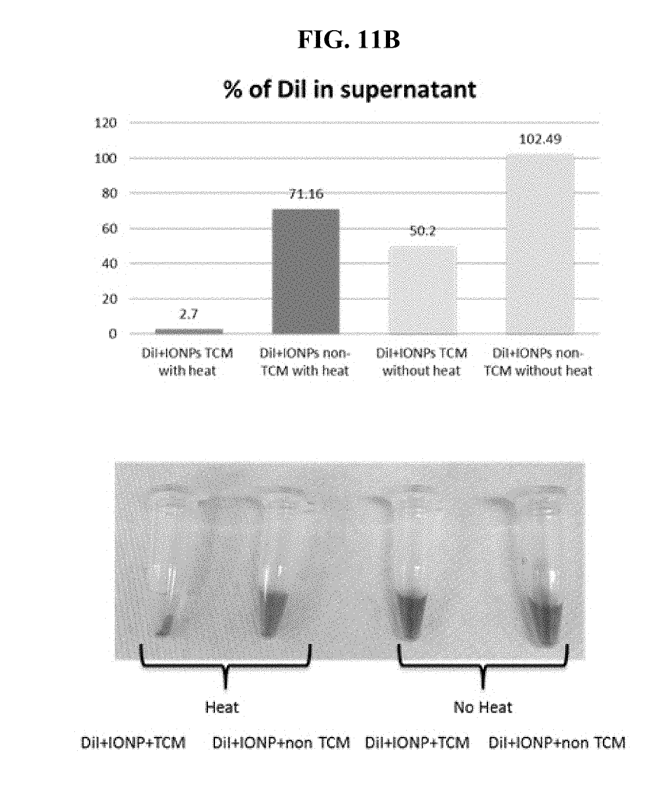

Generating Ball-Like Structure from Mixtures of Cleavable Micelles FIG. 11A shows that DiI-IONP ball-like micelles are formed from the combination of two different encapsulated particles in TCM micelles. IONPs-loaded TCM micelles are mixed with DiI-loaded TCM micelles (DiI is a dye with CAS number 41085-99-8) in an aqueous media and subsequently exposed to heat treatment. IONPs form a ball-like structure with encapsulated DiI dye inside the core of the ball-like micelles. The IONPs aligned at the periphery of the hydrophobic polymer can help prevent the leakiness of DiI from the micelles leading to the reduction of premature release and the increase of the stability of the DiI dye (or other agent) in micelles. Therefore, the ball-like structure could be used as an effective drug or dye carrier for treatment of disease (e.g., cancer treatment) and diagnosis. FIG. 11B shows as chart that shows the percent of DiI dye in supernatant measured by UV absorbance from the solution shown in the lower panel. The lower left picture demonstrates that DiI dye molecules are encapsulated within the ball-like structure as the DiI dye precipitate down together with IONPs after centrifugation at high speed. In contrast, the mixture of DiI-loaded non-TCM and IONPs-loaded non-TCM cannot form the DiI-IONPs loaded ball structure even after heat treatment. Each kind of micelle is still in water separately because the IONPs-loaded non TCM precipitate down as can be seen by the black pallets at the bottom of the tube, while the DiI-loaded non TCM are still suspended in water as can be seen in the pink solution. The lower right picture shows that without heat exposure, DiI-IONPs ball-like structure cannot be effectively formed from TCM and cannot be formed at all from non-TCM.

According to the Example above, it is apparent that during the process of the ball-like structure formation, hydrophobic drugs or dyes (or other agents) can be encapsulated inside the ball-like structure. Consequently, such ball-like structures could be used, for example, as a drug or dye carrier for disease treatment (e.g., cancer treatment) and/or detection. For example, if NIR fluorescence dye is loaded into the ball-like TCM, these nanoparticles could be used for both photothermal therapy and optical imaging because NIR fluorescence dyes can absorb the light at near-infrared region and then convert into heat energy as well as IONPs and gold nanoshells. Moreover, NIR dyes have been reported for in vivo tumor imaging for tumor detection and could be used for such (See, Kim et al. Pharm. Res. 27, 1900-13 (2010); Luo et al., Biomaterials 32, 7127-38 (2011); Ma et al., Biomaterials 34, 7706-14 (2013); and Rodriguez et al., J. Biomed. Opt. 13, 014025 (2014); all of which are herein incorporated by reference).

Generating Janus Nanoparticles Using Mixtures of Cleavable Micelles

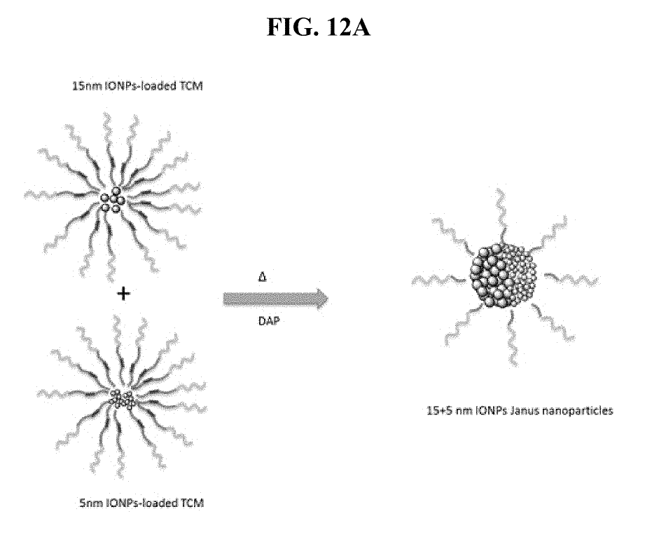

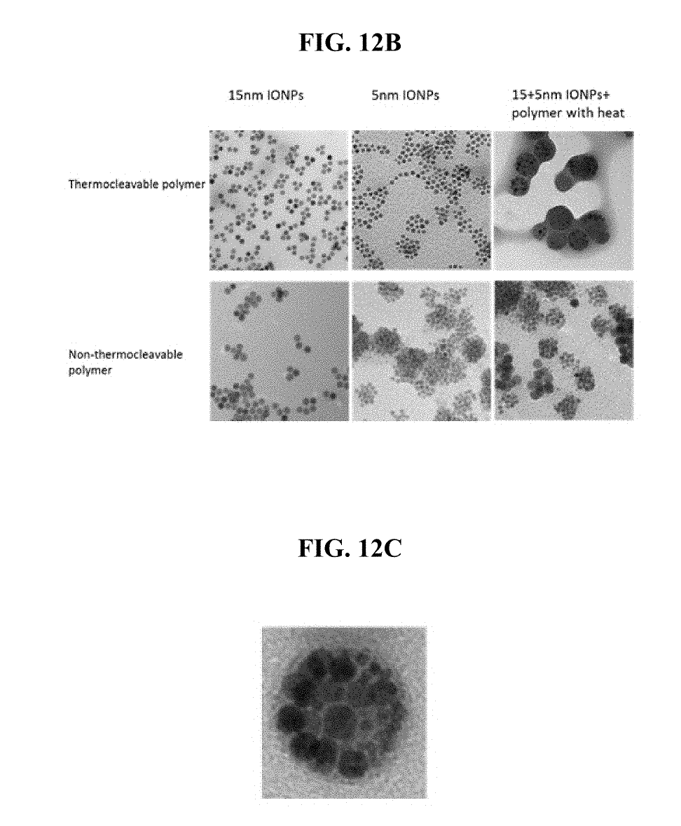

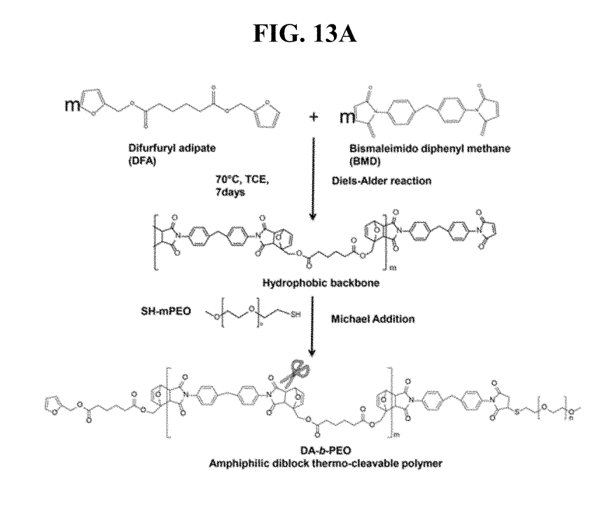

FIG. 12A shows a schematic picture describing an exemplary process for making Janus nanoparticles using mixtures of cleavable micelles. 15 nm IONPs TCM are mixed with 5 nm IONPs TCM and heated up to 94° C. for 2 hours. After the hydrophobic polymer backbone is cleaved by the heat, the two kinds of TCM combine together and generate Janus nanoparticles that generally have 15 nm IONPs on one side/part and 5 nm IONPs on the other side/part. FIG. 12B shows TEM images that demonstrate 15 nm and 5 nm IONPs both in TCM and non-TCM original cluster before heat treatment. However, after being exposed to high temperature, TCM create a new type of micelles, which have both 15 nm and 5 nm IONPs in the same micelles. In contrast, after heat treatment, 15 nm and 5 nm IONP non-TCM are still in separate micelles as the original micelle solution. This confirms that Janus nanoparticles are formed due to the use of the cleavable backbone. FIG. 12C show an image of the Janus nanoparticles at high magnification. The 15 nm IONPs are deposited on the left side of the ball and 5 nm IONPs are deposited at the other.

Janus nanoparticles are a very promising candidate as drug carriers and optical imaging, among other uses. In addition to biomedical applications, Janus nanoparticles can be very useful in the semiconductor industry (see, Walther & Miller, Chem. Rev. 113, 5194-261 (2013), and Reguera et al., Chimia (Aarau). 67, 811-8 (2013); both of which are herein incorporated by reference) as they are composed with two different kinds of elements, which have two different properties. In this Example, a method is demonstrated to produce the Janus nanoparticles by using 15 nm and 5 nm IONPs as examples. Nevertheless, other kinds of elements, and sizes, could be used such as gold nanoparticles, quantum dots, or polymeric nanoparticles.

### Example 3

### Multi-Building Block Janus and Ball-Like Nanostructures Synthesized by Seed-Mediated Self-Assembly from Nanoparticle-Loaded Thermo-Cleavable Polymeric Micelles

This Example describes methods to prepare multi-building block Janus nanoparticles composed of two different inorganic nanoparticles and ball-like nanostructures using thermo-cleavable amphiphilic diblock copolymer to control the nanoparticle distribution and self-assembly of nanoparticles loaded in thermo-cleavable micelles. The thermo-cleavable amphiphilic diblock copolymer, in which the hydrophobic backbone could be cleaved apart at high temperature, was synthesized via retro Diels-Alder reaction resulting in hydrophobic chain shortening and a structural transformation. Gold nanoparticles (AuNPs) and iron oxide nanoparticles (IONPs) were used as examples for multi-building block Janus nanostructure (JNS) formation based on micelle collision and fusion mechanism. A similar strategy was used to generate ball-like nanostructures (BNS), which have an internal void space for carrying hydrophobic drug/dye for theranostics. This method for multi-building block Janus and ball-like nanostructure formation is simple yet efficient. Therefore, using this method, which controls the location and self-assembly of nanoparticles in the thermo-cleavable polymer to form multi-building block Janus and ball-like nanostructures, can serve as a platform to fabricate different JNS compositions for biomedical applications and drug delivery.

### Materials and Methods

### Synthesis of Difurfuryl Adipate (DFA):

Difurfuryl adipate was synthesized by using the previously published method³⁷. Briefly, 5.5 mmol adipoyl chloride was added dropwise to 11 mmol furfuryl alcohol with a few drop of triethanolamine (TEA) in dichloromethane. The reaction was carried out at 0° C. for 3 hours under nitrogen atmosphere. The product was purified by silica gel column chromatography eluted with petroleum ether and ethyl acetate (2:1). The final product was brown viscous liquid. The chemical structure was confirmed by ¹HNMR spectroscopy (Varian 400 MHz) in dichloromethane (DCM)-d₄ and tetrachloro ethane (TCE)-d₂.

### Synthesis of Cleavable Hydrophobic Backbone Polymer, Diels-Alder Polymer (DA):

The DA polymer was synthesized from difufuryl adipate (DFA) and bismaleimido diphenyl methane (BMD) monomers following previously literature^{38,39}. An equimolar of DFA and BMD was mixed in TCE and the reaction was carried out at 70° C. for 7 days. The viscous yellow liquid was precipitated by excess petroleum ether and pale yellow powder was obtained. The powder was dried out under vacuum condition. The final product was characterized by using ¹HNMR spectroscopy in TCE-d₂. Molecular weight of DA was measured by gel permeation chromatography (GPC).

### Cycloadduct Conversion:

in order to determine the percent of cycloadduct conversion, DFA and BMD were reacted at 70° C. for 48 hours to induce Diel-Alder reaction. While retro Diels-Alder happened at 100° C. leads to the cycloadduct cleavage. The percent of cycloadduct conversion was calculated from the area under the peaks appeared in ¹H NMR at 5.32 ppm and 7.43 ppm, which indicates cycloadducts in hydrophobic backbone and furan ring in the starting material respectively.

% Conversion={AUC at 5.32 ppm/[AUC at 5.32 ppm+AUC at 7.43 ppm]}×100

### Synthesis of Thermo-Cleavable Polymer (DA-b-PEO) Via Michael Addition.

The excess molar concentration of thiol-methoxy polyethylene oxide, molecular weight 5,000 Da (SH-mPEO), was added into the solution of DA polymer (1.5:1 molar ratio) in DCM with a few drops of TEA. The solution was kept under stirring for an overnight and the product was precipitated into petroleum ether. The polymer structure was confirmed by using ¹H NMR spectroscopy and molecular weight was determined by GPC.

### Synthesis of IONPs:

15 nm IONPs were synthesized by using previously reported methodo⁴⁰. Briefly, a mixture of 0.890 g FeO(OH), 19.8 g oleic acid and 25.0 g 1-octadecene in a three-neck flask were heated under stirring to 200° C. under N₂, 30 minutes later the temperature was set at 220° C. for 1 h, then the temperature was increased gradually to 310° C. (20° C./5 minutes) and kept at this temperature for 1 h. The solution became black when the temperature was increased to 320° C. and kept at this temperature for 1 h. After the reaction was completed, the reaction mixture was cooled and the nanocrystals were precipitated by adding chloroform and acetone.

### Nanoparticles/Dye Encapsulation in Thermo-Cleavable Micelles:

To make IONP-loaded thermo-cleavable micelles (FeTCM), 4 mg of oleic acid-coated IONPs (15 nm) and 40 mg of DA-b-PEO were dissolved in tetrahydrofuran (THF). The solution was transferred dropwise into water under vigorous agitation. The solution was open to the air overnight to evaporate THF. Free micelles were removed by weight separated centrifugation twice. The similar method was used to make gold-loaded thermo-cleavable micelles (AuTCM). 4 mg of dodecanethiol-coated AuNPs (5 nm) and 40 mg of DA-b-PEO were homogeneously mixed in THF and transferred dropwise into water. Non-thermo-cleavable control micelles (Polystyrene-b-polyethylene oxide, PS-b-PEO, Mw 10,300 Da) were also used to make nanoparticle-loaded non thermo-cleavable micelles by the similar method. To make DiI-loaded thermo-cleavable micelles (DTCM), 2 mg of DiI were mixed with 20 mg of DA-b-PEO in THF and transfer dropwise into water to make DTCM. DTCM was purified by membrane filtration (Amicon Ultra, MWCO, 10,000 Da, Millipore, USA) to remove excess DiI in water.

### Au/IONP Janus Nanostructure (JNS) Formation:

FeTCM (2.7 nM), AuTCM (0.2 M), and DA-b-PEO (10.0 M) were mixed together at room temperature. The solution was subsequently heated at 94° C. for 3 hours in a heat block. The obtained JNS were centrifuged twice to remove free polymer seeds. The JNS solution was kept at 4° C. in the magnet separator (EasySep, USA) for an overnight to remove unreacted AuTCM.

### Synthesis IONP and AuNP Ball-Like Nanostructures:

To form iron oxide ball-like nanostructures, FeTCM (0.54 nM) were homogeneously mixed with TCM seed (0.2 μM) at room temperature. The ball-like formation was carried out at 95° C. for 2 hours in a heat block. Then the solution was centrifuged to remove free TCM seed and the pallets of ball-like nanostructures were redispersed in Milli Q water. To form gold ball-like nanostructures, AuTCM (0.4 μM) was mixed with free TCM seed (10.0 M). The solution was heat at 94° C. for 3 hours in a heat block and purified by centrifugation. The final products were stored at 4° C.

### Synthesis of Scramble Au/IONP Micelles (SCM):

Oleic acid coated-IONPs (1 mg) and of dodecanethiol AuNPs (1 mg) were dissolved together in THF and subsequently added into 20 mg of DA-b-PEO solution in THF under stirring. The resulting solution was slowly dropped into water under vigorous agitation. The product was purified twice by weight separated centrifugation.

### Characterization of Nanoparticle-Loaded Micelles, BNS, and JNS:

NP-loaded TCM, ball-like, and JNS samples for TEM imaging were prepared by the solvent evaporation method. Briefly, the solution (5 μL) of each sample were dropped onto carbon-coated copper TEM grids and allowed to dry overnight. TEM images were acquired on a transmission electron microscope (TEM, Phillips CM-100, 60 kV). Scanning transmission electron microscopy (STEM) and X-ray energy dispersive spectroscopy (XEDS) were performed using Jeol JEM-2010F, operating at 200 kV with a double tilt holder. Images and size distributions were analyzed by ImageJ software from NIH and Gatan digital micrograph software. Hydrodynamic diameters were measured by using Malvern Zeta Sizer Nano S-90.

### Results

### Thermo-Cleavable Polymer Synthesis.

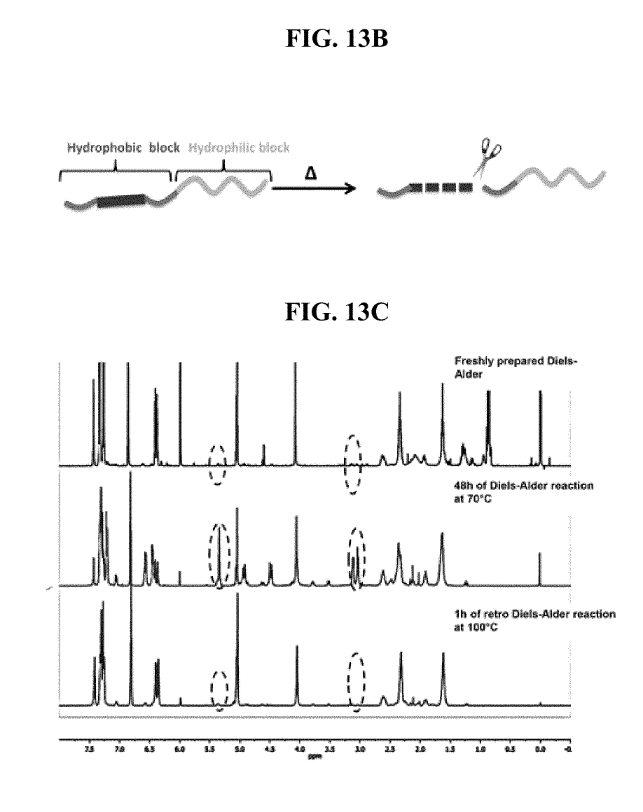

First, in order to generate JNS from nanoparticle-loaded polymeric micelles by the seed mediated self-assembly process, we synthesized thermo-cleavable amphiphilic diblock copolymer (Da-b-PEO) with the molecular weight of 9,800 Da via Diels-Alder reaction at 70° C.^{20,21} and Michael addition (FIG. 13a). This polymer acts as an important component to control hydrophobic interaction between nanoparticles and the polymer itself as well as mediate the micelle fusion. ¹H NMR (400 MHz, TCE-d₂, 25° C., TMS) peaks confirms the thermo-cleavable cycloadducts at δ 7.30 (d, J=8 Hz, 4H), δ 7.19 (d, J=8 Hz, 4H), δ 6.55 (d, J=6.4 Hz, 2H), δ 6.43 (d, J=5.6 Hz, 2H), δ 5.32 (s, 2H), δ 4.91 (d, J=13.2 Hz, 2H), δ 4.47 (d, J=13.2 Hz, 2H), δ 4.04 (s, 2H), δ 3.09 (d, J=5.6 Hz, 2H), δ 3.02 (d, J=6.4 Hz, 2H), δ 2.34 (m, 4H), δ 1.63 (m, 4H) (FIG. 13c). The cycloadducts in hydrophobic backbones could be cleaved at 90° C. or above via retro Diels-Alder reaction²² (FIGS. 13b and 13c). The retro Diels-Alder causes hydrophobic backbone shortening and hydrophobic-hydrophilic imbalance, which affects the thermodynamic stability and triggers self-assembly of the thermo-cleavable micelles (TCM).

### Multi-Building Block Gold/Iron Oxide Janus Nanostructures (JNS) Formation.

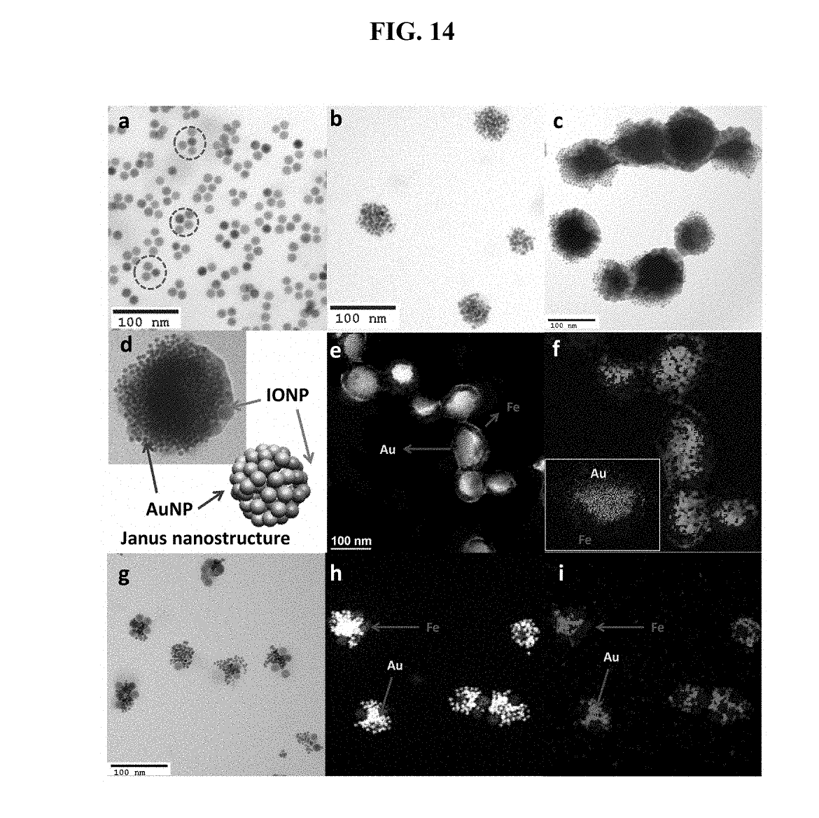

Oleic acid capped iron oxide nanoparticles (15 nm) (IONPs) and dodecanethiol capped gold nanoparticles (5 nm) (AuNPs) were encapsulated separately in Da-b-PEO to make IONP-loaded thermo-cleavable micelles (FeTCM) and AuNP-loaded thermo-cleavable micelles (AuTCM). Transmission electron microscopy (TEM) images clearly showed that nanoparticles aggregated at the center of the hydrophobic domain of the micelles (FIGS. 14a and 14b) due to strong interparticle Van Der Waals attractions. It is noteworthy that we did not observe any ball-like structure or other particular structures in the nanoparticle-loaded micelles (NP-TCM) at this stage. TEM data suggest that FeTCMs and AuTCMs have average diameters of 39.6 nm, and 56.6 nm respectively. Then the excess molar concentrations of AuTCM were homogeneously mixed with FeTCM and together with free TCM seeds followed by the heat treatment (94° C.) to generate asymmetrical Au/IONP JNS. The excess molar concentration of AuTCM was used to ensure that all FeTCMs were fused with AuTCMs. The final solution was purified by a magnetic separator to remove the unreacted AuTCMs. Transmission electron microcopy (TEM) (FIGS. 14c and d) and scanning transmission electron microscope, high angle annular dark-field (STEM-HAADF) (FIG. 14e) images illustrate that AuNPs and IONPs are combined together in a new single entity with a well-defined asymmetrical nanostructure regardless of their orientation shown in TEM and STEM-HAADF images. The average diameter of JNS is 86.5 nm. It is important to note that JNS formed by the self-assembly approach have a relatively small size, sub-100 nm, compared to Janus particles made by other conventional methods, which mostly yield the particles in micron scale single domains²³. We performed an x-ray energy dispersive spectroscopy (XEDS) element mapping to confirm that the JNS are composed of multiple AuNPs and IONPs in an asymmetrical pattern (FIG. 14f).

To prove that thermo-cleavable polymer is an important factor to form JNS, we used Polystyrene-b-polyethylene oxide (PS-b-PEO, Mw 10,300 Da) as a control for non-thermo-cleavable micelles (non-TCM). 15 nm IONPs and 5 nm AuNPs were also encapsulated separately. There was neither self-assembly nor JNS formation after adding PS-b-PEO seeds and high temperature trigger. These two types of nanoparticle-loaded micelles remained in separation. We further ruled out the possibility that different capping ligands of AuNPs and IONPs cause a non-specific asymmetrical structure inside of TCM in an independent manner of polymer properties and self-assembly process, AuNPs and IONPs were homogeneously mixed together at the beginning and subsequently loaded into the thermo-cleavable polymer to form micelles. TEM, STEM-HAADF imaging, and XEDS element mapping show that there was no well-organized asymmetrical JNS formation (FIG. 14g, h, and i). AuNPs and IONPs were randomly mixed together in a micelle without any specific pattern. These data suggested that thermo-cleavable polymer is important for self-assembly and self-reorganization processes to form asymmetrical JNS.

### Ball-Like Nanostructure (BNS) Formation.

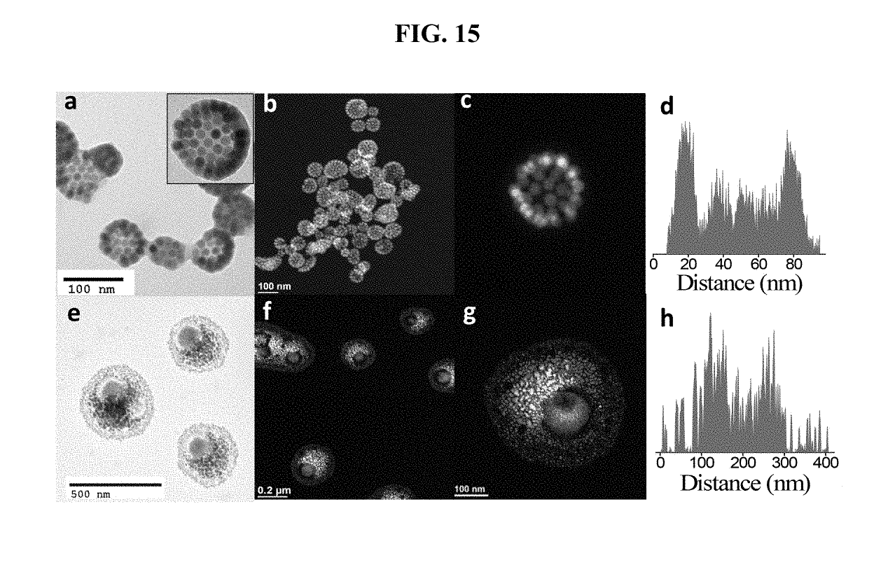

We further developed BNS using the similar method with JNS formation. However, only one species of nanoparticles, either oleic acid capped IONPs (15 nm) (FeTCM) or dodecanthiol capped AuNPs (5 nm) (AuTCM) were mixed with the Free TCM seeds followed by high temperature exposure (94° C.) to disrupt the hydrophobic backbone and trigger self-reorganization of nanoparticle-loaded micelles. TEM images clearly show iron oxide ball-like nanostructures (FeBNS) with diameter ca. 74.1 nm (FIG. 15a). This transformed structure was also confirmed by STEM-HAADF (FIGS. 15b and c). The images and density profiles of FeBNS (FIG. 15d) clearly indicate that the electron density was higher at the edges and lower inside the cores because nanoparticles aligned at the interface between hydrophilic PEG and hydrophobic residues after self-assembly. Interestingly, the self-assembly and transformation of nanoparticle-loaded TCM to ball-like nanostructures are independent of the nanoparticle types. It was noted that the self-assembly and transformation also took place with AuTCM. TEM and STEM-HAADF images clearly show gold ball-like nanostructures (AuBNS) with the diameter of 281.0 nm (FIG. 15e, f, and g). The density profile also confirmed an internal void volume of AuBNS (FIG. 15h). These data suggest that the self-assembly and self-reorganization process has transformed cluster NP-TCMs to ball-like nanostructures. It is important to note that we did not observe the self-reorganization and transformation using the IONP-loaded in PS-b-PEO micelles with the non thermo-cleavable polymer (PS-b-PEO) seed.

We next proved that the core of BNS is capable of encapsulating hydrophobic small molecules such as drug/dye. The hydrophobic dye, 1,1′-Dioctadecyl-3,3,3′,3′-Tetramethylindocarbocyanine Perchlorate (DiI), was used as an example of small hydrophobic molecules to be encapsulated in the core. DiI-loaded TCM (DTCM) were simply mixed with FeTCM followed by high temperature treatment. It was shown that DTCM fused with FeTCM to from ball-like nanostructures with DiI encapsulated inside the core after high temperature treatment. These data suggest that the core inside BNS can encapsulate hydrophobic molecules for drug delivery or imaging.

### Seed-Mediated Self-Assembly Mechanism.

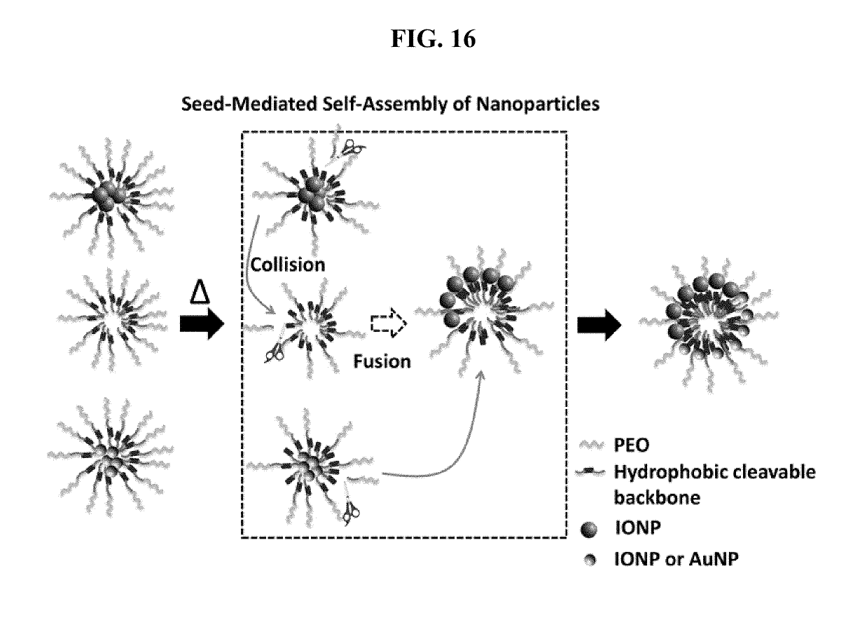

We further investigated the mechanism of JNS and BNS formation. It is clear that the self-assembly and transformation are driven by thermodynamic force. To initiate self-assembly and micelle fusion of these FeTCMs and AuTCMs, the hydrophobic interaction between the polymer backbone itself and nanoparticles needs to be diminished. While the present invention is not limited to any particular mechanism, and an understanding of the mechanism is not necessary to practice the invention, the following is believed. When the mixture of NP-TCMs and free TCM seeds in aqueous media is exposed to the high temperature (94° C.), the hydrophobic backbones in all micelle species are subsequently cleaved apart via retro Diels-Alder reaction resulting in a reduction of the hydrophobic attraction between the backbone and nanoparticles. At this state, NP-TCMs become unstable and relatively flexible. As a consequence, these unstable NP-TCMs try to minimize their interfacial energy and avoid the release of hydrophobic payloads (such as NPs and cleaved polymer backbone residues) by fusing with free TCM seeds in the aqueous media. While one NP-TCM collides with the seed, another NP-TCM can also fuse with the same seed from the opposite side (FIG. 16). The free TCM not only acts as a seed to mediate the self-assembly but also enhance a depletion force between two NP-TCMs^{28,29}. This leads to the structural transformation and self-assembly to form JNS. If there is only one kind of NP-TCMs (either AuTCMs or FeTCMs) mixed with free TCM seeds in the system, ball-like nanostructures will be formed instead of JNS. In contrast, the system without free TCM seed failed to transform into ball-like nanostructures after a high temperature trigger. It was noticed that the hydrophobic IONPs were released out and precipitated into the aqueous media instead (FIG. S9). In the lack of free TCM seed, there is neither a template for NP-TCM to be anchored nor a depletant to attract NP-TCMs; consequently, fusion process is not possible.

After heat treatment, the nanoparticles reorganize themselves and localize at the amphiphilic polymer interface as a ball-like structure. It has been reported that the relation between lengths of polymeric micelles and nanoparticle diameters is one of the significant factors to control over the location of nanoparticles inside micelles^{14,15}. In general, the energy penalty increases with the increasing ratio between nanoparticle sizes and the coil dimension of the polymer. Since the radius of gyration of the nanoparticles is larger than the radius of gyration of the polymer after the backbone cleavage, the nanoparticles will be expelled from the matrix and large-scale phase separation occurs³⁰⁻³². Meanwhile, the small hydrophobic monomers after being cleaved from the hydrophobic backbones, such as difurfuryl adipate and bismaleimido diphenyl methane, have an interaction among themselves and pack densely at the center of the core. This could also expel nanoparticles to the interface to create a space for these small hydrophobic molecules.

### DISCUSSION

In this Example, it was hypothesize that hydrophilic-hydrophobic imbalance and the reduction of the hydrophobic interaction between the nanoparticles and the hydrophobic backbones of the polymer may drive the reorganization of spatial distribution and nanoparticle self-assembly resulting in micelle collision and fusion and subsequently form Janus or ball-like nanostructures to subside overall energy penalty. We used temperature to control over the hydrophobic-hydrophilic interaction of the polymer via Diels-Alder and retro Diels-Alder reaction. It was shown that the temperature-controlled bottom up self-assembly could form an asymmetrical JNS composed of two different types of multi-building block inorganic nanoparticles, which is a complex type of Janus particles. Previously, Hu and Gao generated nanocomposites with spatially separated between iron oxide nanoparticles and Poly (styrene-b-allyl alcohol)⁶. Liu and coworkers demonstrated asymmetrical hybrid vesicles created by a self-assembly between thiol-polystyrene-b-polyethylene oxide polymer and citrate-coated AuNPs²⁸. Although these asymmetrically separated nanocomposites between inorganic nanoparticles and polymers have been reported recently, to our knowledge, our seed-mediated self-assembly method demonstrates, for the first time, to fabricate an asymmetrically separated of two different inorganic nanoparticles as building blocks. This unique nanostructures provide superior properties to other single domain Janus particles because of the high surface-to-volume ratio. The tumor accumulation of nanostructures could be rapidly manipulated by an external magnetic field. The surface plasmon resonance (SPR) of gold nanoparticles is also affected by the interparticle interaction. This interaction enhances non-linear optical properties, which are useful for optical imaging such as surface enhanced Raman scattering. SPR could be fine tuned, for example, by the varying the aggregation number and size of AuNPs in JNS^{33,34}.

Not only is the surface-to-volume ratio important to the biomedical applications, but also the size of a nanoparticle is critical. Most passive targeted nanoparticles take advantages of enhanced permeability and retention (EPR) effect of leaky blood vessels and an impaired lymphatic drainage to reach and accumulate at tumors. However, EPR effect provides the best benefit for the nanoparticles with the size smaller than 150 nm³⁵. Our multi-building block Janus nanostructures have sub-100 nm in diameter, which is small enough to reach the tumors by EPR and prevent uptake by liver and spleen, but big enough to avoid rapid renal clearance³⁶. On the contrary, micron scale single domain Janus particles made by surface masking and phase separation technics are barely used in biomedical applications because of the size limitations.

Ball-like nanostructures are a great candidate for a therapeutic carrier. Ball-like nanostructures provide a better protection for the loaded therapeutic molecules against the external environments such as pH or enzymes in blood stream compared to drugs conjugated at the nanoparticles surface. Moreover, the secondary nanostructures composed of multiple tiny building block nanoparticles are easily to be degraded and excreted from the body compared to the single nanoparticles with the same size²⁷. This minimizes the safety concerns for biomedical usage.

In conclusion, we report a fabrication of asymmetrical multi-building block Janus and symmetrical ball-like nanostructures using the novel self-assembly approach with the thermo-cleavable polymer to control the location and self-assembly of nanoparticles. The seed-mediated collision and fusion proposed mechanisms during the cleavage process of thermo-cleavable polymer are important for multi-building block Janus and ball-like nanostructure formation. The self-reorganization and self-assembly of nanoparticles inside of TCM mitigate the overall energy penalty and increase their stability. The formed multi-building block Janus and ball-like nanostructure could be used, for example, for theranostics, drug delivery, and imaging.

Furthermore, this method and thermo-cleavable polymer provide a new platform for fabrication of nano scale complex Janus and ball-like structures with combinatorial nanocomposites.

### References for Example 3

1. Kaewsaneha, et al., ACS Appl. Mater. Interfaces 5, 1857-69 (2013).
2. Perro, A., et al., J. Mater. Chem. 15, 3745 (2005).
3. Tran, et al., Expert Opin. Drug Deliv. 11, 1061-74 (2014).
4. Bhaskar, S. et al., Small 7, 812-9 (2011).
5. Du & O'Reilly, Chem. Soc. Rev. 40, 2402-2416 (2011).
6. Hu, & Gao, J. Am. Chem. Soc. 132, 7234-7 (2010).
7. Reguera, et al., Chimia (Aarau). 67, 811-8 (2013).
8. Yan, et al., ACS Nano 4, 913-920 (2010).
9. Jiang. et al., Adv. Mater. 22, 1060-71 (2010).
10. Lattuada & Hatton, Nano Today 6, 286-308 (2011).
11. Lattuada, & Hatton, J. Am. Chem. Soc. 129, 12878-12889 (2007).
12. Wurm & Kilbinger, Angew. Chem. Int. Ed. Engl. 48, 8412-21 (2009).
13. Hickey et al., ACS Nano 8, 495-502 (2014).
14. Lamarre, et al., Langmuir 29, 10891-8 (2013).
15. He, J. et al. Self-assembly of amphiphilic plasmonic micelle-like nanoparticles in selective solvents. J. Am. Chem. Soc. 135, 7974-84 (2013).
16. Kanahara, et al., Soft Matter 10, 275-80 (2014).
17. Hickey, et al., ACS Nano 7, 5824-33 (2013).
18. Zubarev, et al., J. Am. Chem. Soc. 128, 15098-9 (2006).
19. Zhang, et al., Nano Lett. 3, 1341-1346 (2003).
20. Goussé, et al., Polym. Int. 48, 723-731 (1999).
21. Gandini, Prog. Polym. Sci. 38, 1-29 (2013).
22. Hall, et al., Polym. Int. 60, 1149-1157 (2011).
23. Grzelczak, et al., ACS Nano 4, 3591-3605 (2010).
24. Luo & Eisenber Langmuir 17, 6804-6811 (2001).
25. Owen et al., Nano Today 7, 53-65 (2012).
26. Deng et al., Adv. Mater. 27, 3645-3653 (2015).
27. Deng et al. Theranostics 4, 904-918 (2014).
28. Liu et al., J. Am. Chem. Soc. 136, 2602-2610 (2014).
29. Nikolic et al., Chemie—Int. Ed. 48, 2752-2754 (2009).
30. Mackay et al., Science 311, 1740-1744 (2006).
31. Thompson et al., Science 292, 2469-2473 (2001).
32. Walther et al., ACS Nano 2, 1167-1178 (2008).
33. Ghosh et al., Chemical Reviews 107, 4797-4862 (2007).
34. Saha et al., Chem. Rev. 112, 2739-2779 (2012).
35. Petros & DeSimone, Nat. Rev. Drug Discov. 9, 615-27 (2010).
36. Dreaden et al., Ther. Deliv. 3, 457-478 (2012).
37. Kuramoto et al., Polym. Sci. 2501-2504 (1994).
38. Gandini et al., J. Mater. Chem. 19, 8656 (2009).
39. Gandini et al., Eur. Polym. J. 44, 4029-4036 (2008).
40. Chen, H. et al., Nanotechnology 24, 355101 (2013).

### References (for all the Above, Except Example 3)

1. Rösler et al., Adv. Drug Deliv. Rev. 64, 270-279 (2012).
2. Fitzpatrick et al., Expert Rev. Med. Devices 9, 339-51 (2012).
3. Wei et al., Prog. Polym. Sci. 38, 503-535 (2013).
4. Cellesi et al., Ther. Deliv. 3, 1395-1407 (2012).
5. Ward & Georgiou. Polymers (Basel). 3, 1215-1242 (2011).
6. Blanco et al., Exp. Biol. Med. (Maywood). 234, 123-31 (2009).
7. Wei et al., Polymer (Guildf). 50, 2836-2840 (2009).
8. Strong & West, Wiley Interdiscip. Rev. Nanomed. Nanobiotechnol. 3, 307-17 (2011).
9. Minchinton & Tannock, Nat. Rev. Cancer 6, 583-92 (2006).
10. Huo et al., Cancer Res. 73, 319-30 (2013).
11. Cabral et al., Nat. Nanotechnol. 6, 815-23 (2011).
12. Wong et al., Proc. Natl. Acad. Sci. 108, 2426-2431 (2011).
13. Nicolaou et al., Angew. Chem. Int. Ed. Engl. 41, 1668-98 (2002).
14. Kuramoto et al., Polym. Sci. 2501-2504 (1994).
15. Gandini, Prog. Polym. Sci. 38, 1-29 (2013).
16. McElhanon et al., Langmuir 21, 3259-66 (2005).
17. Yamashita et al., Langmuir 27, 14621-6 (2011).
18. Gupta & Gupta, Biomaterials 26, 3995-4021 (2005).
19. Chen et al., J. Mater. Chem. B 2, 757 (2014).
20. Tian et al., Small 10, 1063-8 (2014).
21. Chu et al., Biomaterials 34, 4078-88 (2013).
22. Gandini, A. Progress in Polymer Science The furan/maleimide Diels-Alder reaction: A versatile click-unclick tool in macromolecular synthesis. 38, 1-29 (2013).
23. Gandini et al., Eur. Polym. J. 44, 4029-4036 (2008).
24. Cheon et al., J. Am. Chem. Soc. 126, 1950-1 (2004).
25. Starmans et al., PLoS One 8, e57335 (2013).
26. Fu & Kao, J. Biomed. Mater. Res. A 98, 201-11 (2011).
27. Mather et al., Prog. Polym. Sci. 31, 487-531 (2006).
28. Gandini et al., J. Mater. Chem. 19, 8656 (2009).
29. Coelho, D. Novel furan polymer materials based on the reversible Diels-Alder reaction. (2011).
30. Kim, D., Lee, E. S., Park, K., Kwon, I. C. & Bae, Y. H. Doxorubicin loaded pH-sensitive.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of generating Janus nanostructures comprising:
a) combining a first aqueous solution and a second aqueous solution to generate a mixture,
wherein said first aqueous solution comprises a plurality of first thermo-cleavable micelles (first TCMs), and wherein said second aqueous solution comprises a plurality of second thermo-cleavable micelles (second TCMs),
wherein each of said first and second TCMs comprises a plurality of amphiphilic polymer molecules, wherein each of said amphiphilic polymer molecules comprises a hydrophilic polymer and a hydrophobic polymer, and wherein said hydrophobic polymer comprises a first region, a second region, and a cleavable Furan-Maleimide adduct which separates said first from said second regions,
wherein each of said first TCMs further comprises first metal nanoparticles coated with an organic hydrophobic coating, and
wherein each of said second TCMs further comprises second metal nanoparticles coated with an organic hydrophobic coating, wherein said second metal nanoparticles are different from said first metal nanoparticles; and
b) exposing said mixture to heat under conditions such that said hydrophobic polymers are cleaved in said plurality of first and second TCMs, thereby causing said plurality of first and second TCMs to combine together to generate a plurality of Janus nanostructures,
wherein each of said Janus nanostructures comprises some of said first and second metal nanoparticles which are arranged in an asymmetrical pattern inside said Janus nanostructures.

2. The method of claim 1, wherein each of said Janus nanostructures further comprises at least one hydrophobic agent.

3. The method of claim 2, wherein said at least one hydrophobic agent comprises one or more therapeutic or diagnostic agents.

4. The method of claim 1, wherein said first and/or second aqueous solution comprises a physiologically tolerable buffer.

5. The method of claim 1, wherein said hydrophilic polymer is selected from the group consisting of: polyalkylene oxides, polyols, poly(oxyalkylene)-substituted diols and polyols, polyoxyethylated sorbitol, polyoxyethylated glucose, poly(acrylic acids) and analogs and copolymers thereof, polymaleic acids, polyacrylamides, poly(olefinic alcohols), polyethylene oxides, poly(N-vinyl lactams), polyoxazolines; polyvinylamines, and copolymers thereof.

6. The method of claim 1, wherein said hydrophobic polymer is selected from the group consisting of: polystyrenes, styrene-butadiene copolymers, polystyrene-based elastomers, polyethylenes, polypropylenes, polytetrafluoroethylenes, extended polytetrafluoroethylenes, polymethylmetacrylates, ethylene-co-vinyl acetates, polymethylsiloxane, polyphenylmethylsiloxanes, modified polysiloxanes, polyethers, polyurethanes, polyether-urethanes, polyethylene terephthalates, and polysulphones.
